(19) United States
(12) Patent Application Publication
(10) Pub. No.: US 2016/0169896 A1
(43) Pub. Date: Jun. 16, 2016

(54) Methods for Predicting and Treating Bone Metastases in Prostate Cancer Patients

(71) Applicants: INSERM (institut NAtional de la Sante et de la Recherche Medicale), Paris (FR); Universite Claude Bernard - Lyon 1, VIlleurbanne Cedex (FR)

(72) Inventors: Edith Bonnelye, Lyon Cedex 08 (FR); Anaies Fradet, Lyon Cedex 08 (FR); Philippe Clezardin, Lyon Cedex 08 (FR)

(21) Appl. No.: 14/436,639

(22) PCT Filed: Oct. 16, 2013
(86) PCT No.: PCT/EP2013/071630

§ 371 Date: Apr. 17, 2015

(30) Foreign Application Priority Data
Oct. 17, 2012 (EP) 2306290

(51) Int. Cl. G01N 33/574; A61K 31/433; C12Q 1/68

(52) U.S. Cl. CPC G01N 33/57407; C12Q 1/6886; A61K 31/433; C12Q 2600/118; C12Q 2600/158; G01N 2333/723

(57) ABSTRACT

The present invention relates to methods for predicting and treating/preventing bone metastases in prostate cancer patients. In particular, the present invention relates to a method for predicting the occurrence of bone metastases in a prostate cancer patient comprising i) determining the level of expression of ERRα in a prostate tumor sample obtained from the patient, ii) comparing the level determined at step i) with a predetermined reference value and iii) concluding that there is a high risk that the patient develops bone metastases when the level determined at step i) is higher than the predetermined reference value or concluding that there is a low risk that the patient develops bone metastases when the level determined at step i) is lower than the predetermined reference value.

### FIELD OF THE INVENTION

The present invention relates to methods for predicting and treating/preventing bone metastases in prostate cancer patients.

### BACKGROUND OF THE INVENTION

Bone metastases are a frequent complication of cancer, occurring in up to 80 percent of patients with advanced prostate cancer (Vela et al. 2007; Weilbaecher et al. 2011). Bone metastases are not a direct cause of death but are associated with significant morbidity such as bone pain, impaired mobility, hypercalcaemia, pathological fracture and spinal cord compression (Roodman. 2004). Although clinically prostate cancer metastases have been associated primarily with osteoblastic lesions, there is also an osteoclastic component to metastatic bone disease (mixed lesions) (Logothetis and Lin 2005; Vela et al. 2007; Weilbaecher et al. 2011). Indeed, for prostate cancer cells to grow in bone, malignant cells alter bone remodeling (formation and resorption) by secreting factors that will directly affect osteoblasts (bone forming cells) and osteoclasts (bone resorption cells). These signaling proteins include the Receptor activator of NF-kB ligand (RANKL) that stimulates osteoclasts differentiation and action while the osteoprotegerin (OPG) that acts as a decoy receptor for RANK (RANKL receptor), inhibits osteoclastogenesis (Boyle, et al. 2003). Therefore the balance between RANKL and OPG, that can be produced both by prostate cancer cells or by osteoblasts under prostate cancer cells secreted factors, is critical in controlling osteoclast activity and osteolysis in bone metastases (Logothetis and Lin. 2005; Vela et al. 2007; Weilbaecher et al. 2011). In addition, monocyte chemotic protein-1 (MCP1) and the transforming growth factor beta TGFβ may also be expressed by the prostate cancer and can have a direct effect on osteoclast formation and activation (Lu et al. 2007; Weilbaecher et al. 2011). On the other side endothelin-1 (ET1), a mitogenic factor for osteoblasts, Fibroblast growth factor 9 (FGF9) and Wnt pathway factors can promote the growth of osteoblasts at metastatic sites and participate in the osteoblastic progression of prostate cancer in bone (Hall et al. 2006; Li et al. 2008). Concerning TGFβ, it can also recruit mesenchymal stem cells and direct them along the osteoblast lineage (Weilbaecher et al. 2011).

Nuclear steroid receptors are transcription factors that comprise both ligand-dependent molecules such as estrogen receptors (ERs) and a large number of so-called orphan receptors, for which no ligand have yet been determined (Benoit, et al. 2006). Three orphan receptors, estrogen receptor-related receptor alpha (ERRα), ERRβ and ERRγ, (NR3B1 NR3B2 and NR3B3, respectively, according to the Nuclear Receptors Nomenclature Committee, 1999), share structural similarities with ERα and ERβ (NR3A1 and NR3A2 respectively) (Benoit et al. 2006), but they do not bind estrogen (Kallen et al. 2004). Sequence alignment of ERRα and the ERs reveals a high similarity (68%) in the 66 amino acids of the DNA binding domain, but only a moderate similarity (36%) in the ligand-binding domain, which may explain the fact that ERRα recognizes the same DNA binding elements as ERs but does not bind estrogen (Giguere et al. 1988). Although ERRα activity is decreased by the synthetic molecule XCT790, no natural ligand has yet been found (Busch et al. 2004).

ERRα is known to regulate fatty acid oxidation and the adaptative bioenergetic response (Huss et al. 2007; Luo, et al. 2003). It is widely expressed in normal tissues (Bonnelye, et al. 1997; Sladek, et al. 1997) but several RNA expression studies show its presence in a range of cancerous cells including breast, prostate, endometrial, colorectal and ovarian tumour tissues (Ariazi et al. 2002; Cavallini, et al. 2005; Cheung, et al. 2005; Gao, et al. 2006; Stein and McDonnell 2006; Sun, et al. 2005; Suzuki, et al. 2004). ERRα was markedly increased in neoplastic versus normal tissues and ERRα-positive tumors (breast, prostate) were associated with more invasive disease and higher risk of recurrences (Ariazi et al. 2002; Cheung et al. 2005; Fujimura, et al. 2007; Stein and McDonnell 2006). ERRα is also highly expressed in skeletal (bone and cartilage) tissues (Bonnelye et al. 2001; Bonnelye et al. 2007). Its expression in osteoprogenitors, proliferating and differentiating osteoblasts in primary rat calvaria cell cultures correlates with its detection in bone in vivo. Moreover, ERRα has been reported to regulate osteoblast (OB) and osteoclast (OC) development and bone formation in vitro (Bonnelye et al. 2001; Bonnelye et al. 2010; Rajalin et al. 2010) and in vivo (Delhon et al. 2009; Teyssier et al. 2009; Wei et al. 2010). Interestingly, ERRα plays was recently shown to play a dual role during breast cancer progression, promoting local tumor growth, but decreasing osteolytic lesions in bone (Fradet et al. 2011).

### SUMMARY OF THE INVENTION

Estrogen receptor related receptor alpha (ERRα) is implicated in prostate cancer and bone development. The inventors showed that elevated ERRα mRNA expression in PC3 prostate carcinomas cells induced osteolytic bone metastases but also new bone formation in animals compared to that observed with parental PC3 cells that only developed lytic lesions. Osteocalcin and bone sialoprotein were up-regulated by osteoblast and several factors that may regulate osteoblasts formation like endothelin 1, Wnt3a, 5a, 10b and 11 were up-regulated in PC3 cells that overexpressed ERRα. On the otherside, monocyte chemotic protein-1 (MCP1), transforming growth factor beta (TGFβ1, β2 and β3), cathepsin k (CK) were up-regulated in PC3 cells that over-expressed ERRα counteracting the osteoprotegerin (OPG) stimulation providing a mechanistic basis for the increased of lytic lesions in vivo. Similarly to bone metastases experiments, growth of PC3-ERRαWT cells was increased compared to PC3 parental cells. Concomitantly, vascular endothelial growth factor (VEGF) and MMP1 were up-regulated in PC3-ERRαWT cells. Finally we found that ERRalpha is positively associated with the expression of the periostin (POSTN) in cancer-associated fibroblasts (CAFs) of prostate cancer suggesting that combined with the up-regulation of several Wnt members (Wnt3a, 5a, 10b, 11) by ERRalpha in the PC3-ERRα cells, ERRalpha may be also implicated in the early phase of metastatic colonization through the WNT/POSTN pathway and therefore participate to the establishment of the metastatic niche. In conclusion, the data provide evidence that ERRα plays a role during prostate cancer progression, promoting bone osteolysis, participating in the osteoblastic component of prostate cancer in bone and establishment of the metastatic niche.

### DETAILED DESCRIPTION OF THE INVENTION

### Methods for Predicting the Occurrence of Bone Metastases

An object of the invention relates to a method for predicting the occurrence of bone metastases in a prostate cancer patient comprising i) determining the level of expression of ERRα in a prostate tumor sample obtained from the patient, ii) comparing the level determined at step i) with a predetermined reference value and iii) concluding that there is a high risk that the patient develops bone metastases when the level determined at step i) is higher than the predetermined reference value or concluding that there is a low risk that the patient develops bone metastases when the level determined at step i) is lower than the predetermined reference value.

As used throughout the present specification, the term “ERRα” has its general meaning in the art and refers to the human estrogen receptor-related receptor a protein.

The method of the invention is particularly suitable for predicting the occurrence of mixed bone metastases (i.e. a mix of osteolytic lesions and bone formation).

In a particular embodiment the prostate tumor sample may result from the prostate tumor resected from the patient. In another embodiment, the prostate tumor sample may result from a biopsy performed in the primary prostate tumour of the patient. The prostate tumor sample can be fresh, frozen, fixed (e.g., formalin fixed), or embedded (e.g., paraffin embedded).

Measuring the expression level of a gene (i.e ERRα) can be performed by a variety of techniques well known in the art.

Typically, the expression level of a gene may be determined by determining the quantity of mRNA. Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR).

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antl1ranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281 :20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927, 069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Puhlication No. 99/26299 (puhlished May 27, 1999). Separate populations of semiconductor nanocrystals can be produced that are identifiable based on their different spectral characteristics. For example, semiconductor nanocrystals can he produced that emit light of different colors hased on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as ³H), metal chelates such as DOTA and DPTA chelates of radioactive or paramagnetic metal ions like Gd3+, and liposomes.

Detectable labels that can he used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can he used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Puhlication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. .1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/0117153.

It will he appreciated by those of skill in the art that by appropriately selecting labeled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labeled with a first hapten, such as biotin, while a second probe that corresponds to a second target sequence can be labeled with a second hapten, such as DNP. Following exposure of the sample to the probes, the bound probes can he detected by contacting the sample with a first specific binding agent (in this case avidin labeled with a first fluorophore, for example, a first spectrally distinct QUANTUM DOT®, e.g., that emits at 585 mn) and a second specific binding agent (in this case an anti-DNP antibody, or antibody fragment, labeled with a second fluorophore (for example, a second spectrally distinct QUANTUM DOT®, e.g., that emits at 705 mn). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6× SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labeled and contacted with the microarray in hybridization conditions, leading to the formation of complexes between target nucleic acids that are complementary to probe sequences attached to the microarray surface. The labeled hybridized complexes are then detected and can be quantified or semi-quantified. Labeling may be achieved by various methods, e.g. by using radioactive or fluorescent labeling. Many variants of the microarray hybridization technology are available to the man skilled in the art (see e.g. the review by Hoheisel, Nature Reviews, Genetics, 2006, 7:200-210).

Expression level of a gene may be expressed as absolute expression level or normalized expression level. Typically, expression levels are normalized by correcting the absolute expression level of a gene by comparing its expression to the expression of a gene that is not a relevant for determining the cancer stage of the patient, e.g., a housekeeping gene that is constitutively expressed. Suitable genes for normalization include housekeeping genes. This normalization allows the comparison of the expression level in one sample, e.g., a patient sample, to another sample, or between samples from different sources.

Alternatively, the expression level of a gene may be determined at the protein level. For example, the prostate tumor sample of the patient may be contacting with a binding partner specific for the protein of interest (i.e. ERRα).

Typically the binding partner is an antibody or an aptamer. Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies of the invention or a fragment thereof can be prepared and isolated using any technique that provides for the production of antibody molecules by continuous cell lines in culture. Techniques for production and isolation include but are not limited to the hybridoma technique; the human B-cell hybridoma technique; and the EBV-hybridoma technique. In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library.

The binding partners of the invention such as antibodies or aptamers, may be labeled with a detectable molecule or substance, such as a fluorescent molecule, a radioactive molecule or any others labels known in the art. Labels are known in the art that generally provide (either directly or indirectly) a signal. As used herein, the term “labeled”, with regard to the antibody or aptamer, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labeling of the probe or antibody by reactivity with a detectable substance. An antibody or aptamer of the invention may be labeled with a radioactive molecule by any method known in the art. For example radioactive molecules include but are not limited radioactive atom for scintigraphic studies such as I123, I124, In111, Re186, Re188. Preferably, the antibodies against the surface markers are already conjugated to a fluorophore (e.g. FITC-conjugated and/or PE-conjugated).

In some embodiments, immunostained slices of the prostate tumor tissue sample may be obtained with an automated slide-staining system by using a labeled binding partner as above described (e.g. an antibody). Immunochemistry (IHC) is a suitable method for quantifying the expression level of a marker in a tissue sample.

Typically, the prostate tumor sample is preferably fixed in formalin and embedded in a rigid fixative, such as paraffin (wax) or epoxy, which is placed in a mould and later hardened to produce a block which is readily cut. Thin slices of material are prepared using a microtome, placed on a glass slide and submitted to immunohistochemistry, for example using an IHC automate such as BenchMark® XT allowing automatic stained slide preparation for implementing the immunohistochemical staining Then after digitalisation of the slices may be used to quantify the level of the marker. Digitalisation of the slices may be made by scan capture, for example with a high resolution Hamamatsu NanoZoomer® 2.0-HT scanner. The mean, median, min and max of the relevant staining intensity of all positive stained cells detected in the tumour sample may be provided. The values and the distribution of the staining intensity can be compared to the predetermined reference value.

Typically, the predetermined reference values used for comparison may consist of a “cut-of” value. The single “cut-off” value permits discrimination between a high and low risk for the occurrence of bone metastases. Practically, high statistical significance values (e.g. low P values) are generally obtained for a range of successive arbitrary quantification values, and not only for a single arbitrary quantification value. Thus, in one alternative embodiment of the method of determining “cut-off” values, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. According to this specific embodiment of a “cut-off” value, low or high risk for the occurrence of bone metastases can be determined by comparing the level determined at step i) with the range of values which are identified. In certain embodiments, a cut-off value thus consists of a range of quantification values, e.g. centered on the quantification value for which the highest statistical significance value is found (e.g. generally the minimum P value which is found). For example, on a hypothetical scale of 1 to 10, if the ideal cut-off value (the value with the highest statistical significance) is 5, a suitable (exemplary) range may be from 4-6. Therefore, a patient may be assessed by comparing values obtained by determining the expression level of ERRalpha, where values greater than 5 indicate a high risk for the occurrence of bone metastases and values less than 5 indicate a low risk; or a patient may be assessed by comparing values obtained by determining the expression level of ERRalpha and comparing the values on a scale, where values above the range of 4-6 indicate a high risk for the occurrence of bone metastases and values below the range of 4-6 indicate a low risk, with values falling within the range of 4-6 indicating an intermediate risk.

### Methods for the Treatment of Bone Metastases

An object of the invention relates to an inhibitor of ERRα activity or expression for use in the treatment of bone metastases in a prostate cancer patient in need thereof.

As used herein, the terms “treatment”, “treating”, and the like, refer to obtaining a desired pharmacologic and/or physiologic effect. In particular, the effect is prophylactic in terms of completely or partially preventing the development of bone metastases in the prostate cancer patient. Accordingly in some embodiments, the patient is considered at risk for the occurrence of bone metastases according to the method of the invention as above described.

An “inhibitor of ERRalpha activity” has its general meaning in the art, and refers to a compound (natural or not) which has the capability of reducing or suppressing the activity of ERRalpha. Typically, said compound inhibits or reduces the transcription from promoters containing ERRalpha binding sites. For example the compound may block the binding of ERRalpha with its natural ligands (e.g. by occupying the binding pocket of ERRalpha), or may block the interaction of ERRalpha with the ERRalpha binding sequences, or may bind to ERRalpha in manner that ERRalpha is not able to bind to the ERRalpha binding sites. Alternatively, said compound may block the binding of ERRalpha with coactivators or may favour the binding of ERRalpha with cosuppressors. Typically, said inhibitor is a small organic molecule or a biological molecule (e.g. peptides, lipid, aptamer . . . ).

In a particular embodiment, the activity of ERRalpha can be reduced using a “dominant negative.” To this end, constructs which encode, for example, defective ERRalpha polypeptide, such as, for example, mutants lacking all or a portion of the DNA binding domain, can be used in gene therapy approaches to diminish the activity of ERRalpha on appropriate target cells. For example, nucleotide sequences that direct host cell expression of ERRalpha in which all or a portion of the DNA binding domain is altered or missing can be introduced in the prostate tissue (either by in vivo or ex vivo gene therapy methods known in the art). Alternatively, targeted homologous recombination can be utilized to introduce such deletions or mutations into the subject's endogenous ERRalpha gene in the prostate tissue. The engineered cells will express non-functional ERRalpha polypeptides.

In a particular embodiment, the inhibitor of ERRalpha activity is an inverse agonist. Examples of inverse agonists include but are not limited to XCT-790 [(2E)-3-(4-{[2,4-bis(trifluoromethyl)benzyl]oxy}-3-methoxyphenyl)-2-cyano-N-[5-(trifluoromethyl)-1,3,4-thiadiazol-2-yl]acrylamide] (Busch B B S W J, Martin R, Ordentlich P, Zhou S, Sapp D W, Horlick R A, and Mohan R. Identification of a selective inverse agonist for the orphan nuclear receptor estrogen-related receptor alpha. 2004 ; J Med Chem 47 (23) 5593-5596). Other inverse agonists also include those described in:
Chisamore M J, Wilkinson H A, Flores O, Chen J D 2009 Estrogen-related receptor-alpha antagonist inhibits both estrogen receptor-positive and estrogen receptor-negative breast tumor growth in mouse xenografts. Mol Cancer Ther 8(3):672-81.
Patch R J, Searle L L, Kim A J, De D, Zhu X, Askari H B, O'Neill J C, Abad M C, Rentzeperis D, Liu J, Kemmerer M, Lin L, Kasturi J, Geisler J G, Lenhard J M, Player M R, Gaul M D 2011 Identification of diaryl ether-based ligands for estrogen-related receptor alpha as potential antidiabetic agents. J Med Chem 54(3):788-808
Fiori J L, Sanghvi M, O'Connell M P, Krzysik-Walker S M, Moaddel R, Bernier M. The cannabinoid receptor inverse agonist AM251 regulates the expression of the EGF receptor and its ligands via destabilization of oestrogen-related receptor a protein. Br J Pharmacol. 2011 October; 164(3):1026-40
Wang J, Fang F, Huang Z, Wang Y, Wong C. Kaempferol is an estrogen-related receptor alpha and gamma inverse agonist. FEBS Lett. 2009 Feb. 18; 583(4):643-7).

An “inhibitor of ERRalpha expression” refers to a natural or synthetic compound that has a biological effect to inhibit or significantly reduce the expression of the gene encoding for ERRalpha.

Inhibitors of expression for use in the present invention may be based on anti-sense oligonucleotide constructs. Anti-sense oligonucleotides, including anti-sense RNA molecules and anti-sense DNA molecules, would act to directly block the translation of ERRalpha mRNA by binding thereto and thus preventing protein translation or increasing mRNA degradation, thus decreasing the level of ERRalpha, and thus activity, in a cell. For example, antisense oligonucleotides of at least about 15 bases and complementary to unique regions of the mRNA transcript sequence encoding ERRalpha can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. ERRalpha gene expression can be reduced by contacting a subject or cell with a small double stranded RNA (dsRNA), or a vector or construct causing the production of a small double stranded RNA, such that ERRalpha gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′- ends of the siRNAs of the invention are also advantageously protected, for example, using the technique described above for protecting the phosphodiester bonds. The siRNAs sequences advantageously comprise at least twelve contiguous dinucleotides or their derivatives.

As used herein, the term “siRNA derivatives” with respect to the present nucleic acid sequences refers to a nucleic acid having a percentage of identity of at least 90% with erythropoietin or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “best alignment” or “optimal alignment”, means the alignment for which the determined percentage of identity (see below) is the highest. Sequences comparison between two nucleic acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p:482, 1981), by using the local homology algorithm developped by NEDDLEMAN and WUNSCH (J. Mol. Biol., vol. 48, p:443, 1970), by using the method of similarities developed by PEARSON and LIPMAN (Proc. Natl. Acd. Sci. USA, vol. 85, p:2444, 1988), by using computer software using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p:1792, 2004). To get the best local alignment, one can preferably used BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of ERRalpha mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing ERRalpha. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV2 (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter. The promoter may be specific for the prostate cell.

The inhibitor of ERRalpha activity or expression may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The Inhibitor of ERRalpha activity or expression of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The inhibitor of ERRalpha activity or expression of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

### Methods for Screening Compounds for the Treatment of Bone Metastases

The present invention also relates to a method for screening a plurality of candidate compounds useful for treating (e.g. preventing) bone metastases comprising the steps consisting of (a) testing each of the candidate compounds for its ability to inhibit ERRalpha activity or expression and (b) and positively selecting the candidate compounds capable of inhibiting said ERRalpha activity or expression.

Typically, the candidate compound is selected from the group consisting of small organic molecules, peptides, polypeptides or oligonucleotides. Other potential candidate compounds include antisense molecules, siRNAs, or ribozymes.

Testing whether a candidate compound can inhibit ERRalpha activity or expression can be determined using or routinely modifying reporter assays known in the art.

For example, the method may involve contacting cells expressing ERRalpha with the candidate compound, and measuring the ERRalpha mediated transcription (e.g., activation of promoters containing ERRalpha binding sites), and comparing the cellular response to a standard cellular response. Typically, the standard cellular response is measured in absence of the candidate compound. A decrease cellular response over the standard indicates that the candidate compound is an inhibitor of ERRalpha activity.

In another embodiment the invention provides a method for identifying a ligand that binds specifically to ERRalpha (e.g. a ligand occupying the binding pocket of ERRalpha without activating it). For example, the putative ligand is incubated with labeled ERRalpha and complexes of ligand bound to ERRalpha are isolated and characterized according to routine methods known in the art. Alternatively, an ERRalpha polypeptide is be bound to a solid support so that binding molecules are bound to the solid support (e.g. a column) and then eluted and characterized according to routine methods.

Another method involves screening for compounds which inhibit ERRalpha activity by determining, for example, the amount of transcription from promoters containing ERRalpha binding sites in a cell that expresses ERRalpha. Such a method may involve transfecting a eukaryotic cell with DNA encoding ERRalpha such that the cell expresses ERRalpha, contacting the cell with a candidate compound, and determining the amount of transcription from promoters containing ERRalpha binding sites. A reporter gene (.e.g, GFP) linked to a promoter containing an ERRalpha binding site may be used in such a method, in which case, the amount of transcription from the reporter gene may be measured by assaying the level of reporter gene product, or the level of activity of the reporter gene product in the case where the reporter gene is an enzyme. A decrease in the amount of transcription from promoters containing ERRalpha binding sites in a cell expressing ERRalpha, compared to a cell that is not expressing ERRalpha, would indicate that the candidate compound is an inhibitor of ERRalpha activity. The expression of others genes such as (VEGF) may also be investigated.

The candidate compounds that have been positively selected may be subjected to further selection steps in view of further assaying its properties on bone metastases. For example, the candidate compounds that have been positively selected with the screening method as above described may be inoculated to an animal model (e.g. SCID mice) with ERRalpha positive prostate cancer cells and then osteolytic lesions are analysed (e.g. radiographic analysis, microtomography), quantified and finally compared to an animal model that was inoculated with ERRalpha positive prostate cancer cells in the absence of the candidate compound. If the candidate is able to induce less osteolytic lesions in the animal the candidate compound may be positively selected for further investigations or pharmaceutical development.

The invention will be further illustrated by the following figures and examples. However, these examples and figures should not be interpreted in any way as limiting the scope of the present invention.

### FIGURES

FIG. 1: Modulation of ERRα in PC3, a prostate cancer cell line highly metastatic to bone. (A) Detection by real-time PCR of ERRα mRNA expression in several prostate cancer cells lines. (B) Confirmation of ERRα protein expression in PC3 cells in vivo by immunohistochemistry in bone metastases present 6 weeks after intra-tibial injection of PC3 cells. (C) Isolation, after stable transfection of the PC3 cell lines; three independent PC3-ERRαWT (WT-1-3) clones and three controls PC3-CT (empty vector) (CT-1-3). ERRα expression was assessed by Western blotting with a monoclonal antibody against ERRα and (D) by real-time PCR on triplicate samples and normalized against that of the ribosomal protein gene L32 (ANOVA, p<0.0001), VEGF expression was increased in PC3-ERRαWT (ANOVA, p<0.0001 for VEGF in PC3-ERRα WT (WT-1-3) versus PC3-CT (CT-1-3)). PCR were performed in triplicate and are representative of two independent experiments. (B) Bar=400 μm. T: Tumor; OB: osteoblasts

FIG. 2: Stimulation of tumor progression and angiogenesis by ERRα in vivo. (A-B) PC3-ERRαWT (pool WT-1-3) or PC3-CT (pool CT-1-3) cells were inoculated subcutaneously of SCID mice. Tumor progression was followed from day 21-42. Greater tumor expansion was observed in mice with PC3-ERRαWT(pool) compared to PC3-CT (pool) cells. Weight and volume of tumors dissected at endpoint are shown (C) Real-time PCR was performed to evaluate VEGF and MMP-1 expression within tumors (pool of n=3 for each condition) correlating with greater tumor progression. ANOVA, p<0.0001 for VEGF and MMPI in PC3-ERRα WT (WT-1-3) versus PC3-CT (CT-1-3)

FIG. 3: Overexpression of ERRα stimulates osteolytic lesions and induce new bone formation in bone metastases. (A, E) PC3-ERRαWT (pool WT-1-3) or PC3-CT (pool CT-1-3) cells were inoculated into SCID mice; 3 weeks post inoculation, radiography revealed larger lesions in mice injected with PC3-ERRαWT cells (n=10) compared to mice injected with CT cells (n=7)(see white arrows) (Osteolysis surface; Mann Whitney, p=0.011). (B, E) Three dimensional microCT reconstructions of tibiae (BV/TV %: Mann-Whitney, p=0.022; Bone formation %: Mann Whitney, p=0.018) and (C, E) histology after Goldner's Trichrome staining (TB/STV %: Mann-Whitney, p=0.008) confirmed the radiography results. (D, E) TRAP (red) staining of osteoclasts (black arrow) in sections of tibiae taken from mice injected with PC3-ERRαWT (pool)) or PC3-CT (pool) cells shows an increase on bone/tumor surface of active osteoclasts in PC3-ERRαWT compared to PC3-CT (Oc.S/BS %: Mann-Whitney, p=0.012). T: Tumor. White arrows indicates osteolytic lesions. Red and black arrows indicates new bone formation and TRAP+osteoclasts respectively.

FIG. 4: ERRα expression in PC3 cells has no impact on OCs formation. (A, B) TRAP (red) staining of OCs (black arrow) in sections of tibiae taken from mice injected with PC3-ERRαWT (pool WT-1-3) or PC3-CT (pool CT-1-3) cells shows an increase on surface of active OCs in PC3-ERRαWT (pool WT-1-3) compared to PC3-CT (pool CT-1-3). (C) Primary mouse bone marrow cells were cultured in the presence of RANKL and M-CSF and treated with conditioned medium obtained from PC3-ERRαWT (pool WT-1-3) (WT) or PC3-CT (pool CT-1-3) (CT) cells. Differently to in vivo, ERRα over-expression in PC3 cells has no direct impact on OCs formed in cultures in vitro suggesting an indirect effect of ERRα over-expression in PC3 cells throught other cell types into the bone environment Bar=400 μm

FIG. 5: ERRα expression in PC3 cells stimulates OBs formation. (A) Primary OB obtain from mouse calvaria cells were treated or not (NT) with conditioned medium by PC3-ERRαWT (pool WT-1-3) (WT) or PC3-CT (pool CT-1-3) (CT) cells. Bone nodules number was increased in PC3-ERRαWT (pool WT-1-3) treated cells compared to PC3-CT (pool CT-1-3) cells (Mann and Whitney, p=0.0118). (B) Concomitantly, MC3T3-E1 cells were treated or not (NT) with conditioned medium by PC3-ERRαWT (pool WT-1-3) (WT) or PC3-CT (pool CT-1-3) (CT) cells for 6 days in osteogenic conditions and osteoblastic markers expression was addressed by real-time PCR. BSP, OCN was found stimulated in presence of PC3-ERRαWT conditioned medium (ANOVA p<0.0001 (BSP) p<0.001 (OCN) while RANKL was decreased (ANOVA p<0.001).

FIG. 6: Regulation of factors implicated into bone osteolysis and bone formation by ERRα in PC3 cells in tumors in vivo. (A) Real-time PCR was performed to evaluate expression of osteolytic factors Runx2, CK, MCP1, and TGFβ within tumors (pool of n=3 for each condition) correlating with greater tumor osteolysis in vivo. ANOVA, p<0.0001 for Runx2, CK, and TGFβ1 in PC3-ERRα WT (WT-1-3) (WT) versus PC3-CT (CT-1-3) (CT). MCP1 only has a tendency to increase but that was not significant. OPG, an inhibitor of osteolysis was also up-regulated in PC3-ERRα WT (WT-1-3) (WT) versus PC3-CT (CT-1-3) (CT) tumors (ANOVA, p<0,0001) (B) Real-time PCR was performed an show an increase in bone formation factors expression Wnt3a, 5a, 10b and 11 and ET1 within the same tumors than (A). ANOVA, p<0.0001 for Wnt3a, 5a, ANOVA, p<0.001 for Wnt11 and ET1 and ANOVA, p<0.05 for Wnt10b. DKK1, an inhibitor of bone formation was also up-regulated in PC3-ERRα WT (WT-1-3) (WT) versus PC3-CT (CT-1-3) (CT) tumors (ANOVA, p<0.0001).

FIG. 7: Modulation of factors implicated into bone osteolysis and bone formation by ERRα in PC3 cells in vitro. (A) Similarly to tumors, Real-time PCR was performed to evaluate expression of osteolytic factors Runx2, CK, MCP1, and TGFβ1 within PC3-ERRα WT (WT-1-3) clones compared to PC3-CT (CT-1-3). Confirming in vivo data, Runx2, CK; TGFβ1 and OPG were up-regulated into PC3 clones over-expressing ERRα. MCP1 was also highly stimulated in WT1 and WT2. As expected OPN was also found up-regulated in clones while it was hard to detect it in tumors in vivo. (B) Similarly, Wnt3a, Wnt10b, ET1 and DKK1 were also found up-regulated into PC3 clones over-expressing ERRα, while Wnt5a and Wnt11 were only stimulated into WT1 clone. Finally FGF9 was also stimulated into PC3 clones over-expressing ERRα in vitro.

FIG. 8: Regulation of ET1 and VEGF by ERRα in bone metastases in vivo. As expected, we show that immunostaining of ERRα (B, C), ET1 (E, F) and VEGF (H, I) are increased in mice bearing WT bone metastases compared to CT after 3 weeks post cell inoculation. Negative controls (CT−) are shown (A, D, G). Bar=400 μm T: Tumor

FIG. 9: Regulation of TGF and OPG by ERRα in bone metastases in vivo. Increased of TGFβ (B, C) and OPG (E, F) immunostaining in mice bearing WT bone metastases compared to CT after 3 weeks post cell inoculation. Negative controls (CT−) are shown (A, D). Bar=400 μm T: Tumor.

FIG. 10: (A) Real-time PCR was performed to evaluate expression of mouse POSTN within tumors (pool of n=3 for each conditions) in vivo. POSTN from stroma was up-regulated in WT (PC3-ERRα WT (WT-1-3)) versus CT (PC3-CT (CT-1-3)) tumors. (B) Concomitantly, increased expression of POSTN was also visualized by immunostaining using mouse POSTN antibody in primary prostate tumors and in bone metastases in vivo in WT tumors versus CT. ***=p<0.001. Overexpression of POSTN was also correlated to the up-regulation of TGFβ2 and 3 expression in WT (PC3-ERRα WT (WT-1-3)) versus CT (PC3-CT (CT-1-3)) tumors suggesting that ERRα may be implicated into the metastatic niche.

FIG. 11: Real-time PCR was performed to evaluate expression of mouse adipokines leptin and adiponectin expression within tumors (pool of n=3 for each conditions) in vivo. Leptin and adiponectin from tumor stroma was down-regulated in WT (PC3-ERRα WT (WT-1-3)) versus CT (PC3-CT (CT-1-3)) tumors. Adipocytes marker FABP4 was also down-regulated confirming the inhibition of adipocyte lineage in the ERRα-PC3 tumor stroma.

FIG. 12 Real-time PCR was performed to evaluate expression of human adipokines leptin and adiponectin expression within tumors (pool of n=3 for each conditions) in vivo. Leptin and adiponectin expression from tumor was down-regulated in WT (PC3-ERRα WT (WT-1-3)) versus CT (PC3-CT (CT-1-3)) tumors. Adipocyte marker PPARγ was not modulated. As previously shown Wnt members (pro-osteoblastic factors) expression was up-regulated suggesting that ERRα in tumor cell may trigger osteoblast phenotype (tumor cell osteomimetism) by down-regulated adipocyte markers and up-regulated osteoblast markers

FIG. 13 Real-time PCR was performed to evaluate expression of cancer stem cells markers within tumors (pool of n=3 for each conditions) in vivo. Nanog and Oct4 expression from tumor was down-regulated in WT (PC3-ERRα WT (WT-1-3)) versus CT (PC3-CT (CT-1-3)) tumors. Sox9 was at the inverse up-regulated

FIG. 14: Visualisation by immunostaining of ERRα expression in primary prostate cancer tumor and associated bone metastases. Left: primary tumors. Right: bone metastases.

FIG. 15. Real-time PCR was performed to evaluate expression of several factors found regulated in vivo in PC3 treated with the inverse agonist XCT-790 compared control (DMSO) for 24 hours. (A) Osteolytic factors Runx2, and MCP1 but no CK were found statistically down-regulated after XCT treatment such as OPG. (B) The osteoforming factors ET1, Wnt3a and Wnt5a were also identified as direct ERRα/PGC1 target genes. *=p<0.05; **=p<0.01; ***=p<0.001. CT versus WT; T test

### EXAMPLE 1

### Implication of ERRα in Mediating Mixed Metastatic Bone Formation From Prostate Cancer Cells

### Material & Methods

### Ethics Statement

The mice used in our study were handled according to the rules of Décret No 87-848 du 19 Oct. 1987, Paris. The experimental protocol have been reviewed and approved by the Institutional Animal Care and Use Committee of the Université Claude Bernard Lyon-1 (Lyon, France). Studies were routinely inspected by the attending veterinarian to ensure continued compliance with the proposed protocols. SCID mice, 6 weeks age, were housed under barrier conditions in laminar flow isolated hoods. Animals bearing tumor xenografts were carefully monitored for established signs of distress and discomfort and were humanely euthanized.

### Animal Studies.

For intra-osseous tumor xenograft experiments (Charles River Laboratories; Wilmington, Mass., USA), a small hole was drilled with a 26-gauge sterile needle through the left tibial plateau with the knee flexed in anesthesized 6- to 8-week-old SCID mice. Using a new sterile needle fitted to a 50-μL sterile Hamilton syringe (Hamilton Co.; Bonaduz, GR, Switzerland), a single-cell suspension (6×10⁵ in 15-μL PBS) of PC3 cells was carefully injected in the bone marrow cavity. On week 2 after tumor cell inoculation, radiographs of anesthetized mice were weekly taken with the use of MIN-R2000 films (Kodak) in an MX-20 cabinet X-ray system (Faxitron X-ray Corp.). Animals were killed after 3 weeks for PC3 model. Microcomputed tomography analyses were carried out using a Skyscan 1174 X-ray-computed microtomograph (Skyscan; Kontich, Belgium), imaged with an X-ray tube (50 kV; 80 μA) and a 0.5 μm aluminum filter. Three-dimensional modeling and analysis of BV/TV ratio (percentage of bone tissue) and bone formation/total bone were obtained with the CTAn (version 1.9, Skyscan) and CTVol (version 2.0, Skyscan) software. The dissected bones were then processed for histological and histomorphometric analysis.

### Cell Lines and Transfection

PC3 cell line was obtained from the American Type Culture Collection (ATCC, Manassas, Va., USA). PC3 cells are routinely cultured in F12K nutrient mixture and DMEM medium (Gibco/Invitrogen; Carlsbad, Calif., USA) respectively supplemented with 10% (v/v) fetal bovine serum (FBS; Perbio/Thermo scientific; Rockford, Ill., USA) and 1% (v/v) penicillin/streptomycin (Invitrogen; Carlsbad, Calif., USA) at 37° C. in a 5% CO2 incubator.

Human ERRα cDNA (WT) was obtained from mRNA extracted from BO2-FRT cells, by using RT-PCR with specific primers ((NM_004451.3) (Fradet et al, 2011). Amplimers were sequenced for verification. The pcDNA5/ERRα-WT (WT) and empty pcDNA5 (used as control (CT)) constructs were transfected using Transfast (Promega). For clonal selection, cells were cultured for 4 weeks in the presence of puromycin (1 μg/ml PC3) (Invitrogen). XCT790, an inverse agonist of ERRα was tested in vitro on PC3 cells at 5.10⁻⁷M compare to DMSO for 24 h.

### Bone Histomorphometry and Histology.

Tibia from animals were fixed, decalcified with 15% EDTA and embedded in paraffin. Five μm sections were stained with Golner's Trichrome and proceeded for histomorphometric analyses to calculate the TB (Tumor burden)/TV ratio (percentage of tumor tissue). The in situ detection of osteoclasts was carried out on metastatic bone tissue sections using the tartarte-resistant acid phosphatase (TRAP) activity kit assay (Sigma). The resorption surface (Oc.S/.BS) was calculated as the ratio of TRAP-positive trabecular bone surface (Oc.S) to the total bone surface (BS) using the computerized image analysis system MorphoExpery (Exploranova). Results are plotted as the mean±SD.

### Osteoclastogenesis Assay

Bone marrow cells from 6-week-old OF1 male mice were cultured for 7 days, in differentiation medium: a-MEM medium containing 10% fetal calf serum (Invitrogen), 20 ng/mL of M-CSF (R&D Systems) and 200 ng/mL of soluble recombinant RANK-L (Fradet et al. 2011). Cells were continuously (day 1 to day 7) exposed to conditioned medium extracted from PC3 clones. After 7 days mature multinucleated osteoclasts (OCs) were stained for tartarte-resistant acid phosphatase TRAP activity (Sigma-Aldrich), following the manufacturer's instructions. Multinucleated TRAP-positive cells containing three or more nuclei were counted as OCs. Results are plotted as the mean±SD.

### Osteoblastogenesis Assay

Cells were enzymatically isolated from the calvaria of 3-day-old OF-1 mice by sequential digestion with collagenase, as described previously (Bonnelye et al. 2008). Cells obtained from the last four of the five digestion steps (populations II-V) were plated onto 24-well plates at 2×10⁴ cells/well. Cells were cultured in α-MEM medium containing 10% fetal calf serum (Invitrogen). After 24 hours incubation, the medium was changed and supplemented with 50 μg/ml ascorbic acid (Sigma-Aldrich). 10 mM sodium β-glycerophosphate (Sigma-Aldrich) was added during 1 week at the end of the culture. Mouse calvaria cells were continuously (day 1 to day 15) exposed to conditioned medium extracted from PC3. For quantification of bone formation, wells were fixed and stained with von Kossa and for ALP and bone nodules were counted on a grid (Bonnelye et al. 2008). Results are plotted as the mean number of nodules±SD of three wells for controls and each conditions (PC3) and were representative of two independent experiments. Similarly to primary osteoblastic cells, MC3T3-E1 cell line, a generous gift of George BOIVIN (Inserm U1033, Lyon) were cultured for 6 days.

### Immunocytochemistry

Metastatic tibia were fixed and embedded in paraffin. Five mm sections were subjected to immunohistochemistry using rabbit polyclonal antibodies for anti human VEGF antibody (Abcam), monoclonal anti human ERRα antibody (Santa Cruz), anti human ET1 antibody (Abbiotec), monoclonal anti human TGFβ1 antibody (R&D systems) and anti human OPG antibody (Abbiotec). Sections were deparaffinized in methylcyclohexan, hydrated then treated with a peroxidase blocking reagent (Dako). Sections were incubated with normal calf serum for 1 hour and incubated overnight at 4° C. with primary antibodies (dilution: 1/50 for ERRα, VEGF, TGFβ1 and 1/200 for ET1, OPG). Sections were incubated with secondary antibody HRP-conjugated donkey anti rabbit (Dako) (dilution 1/300) for 1 hour for VEGF, ET1 and OPG and with secondary antibody HRP-conjugated donkey anti mouse (Dako) (dilution 1/300) for 1 hour for ERRα and TGFβ1. After washing, the sections were revealed by 3,3′-diaminobenzidine (Dako). Counterstaining was performed using Mayer's hematoxylin (Merck).

### Immunoblotting

Cell proteins were extracted, separated in 4-12% SDS-PAGE (Invitrogen), then transferred to nitrocellulose membranes (Millipore) using a semidry system. Immunodetection was performed using a rabbit monoclonal antibody against ERRα (Epitomics, Burligame, Calif.) at a dilution of 1/400 and the secondary antibody (HRP-conjugated donkey anti rabbit) at a dilution of 1/3000 (Amersham). For evaluating protein loading, a rabbit polyclonal antibody against GAPDH (Abcam) was used at a dilution of 1/10000; secondary antibody was used at a dilution of 1/10000 (Amersham). An ECL kit (PerkinElmer) was used for detection.

### Real Time RT-PCR

Total RNA was extracted with Trizol reagent (Sigma) from PC3 cells, PC3 tumors, MC3T3-E1 cells. Samples of total RNA (2 μg) were reverse-transcribed using random hexamer (Promega) and the first strand synthesis kit of Superscript™ II (Invitrogen). Real-time RT-PCR was performed on a Roche Lightcycler Module (Roche) with primers specific for human and mouse. Real-time RT-PCR was carried out by using (SYBR Green; Qiagen,) on the LightCycler system on (Roche) according to the manufacturer's instructions with an initial step for 10 min at 95° C. followed by 40 cycles of 20 sec at 95° C., 10 sec at Tm (see Tables 1-2) and 10 sec at 72° C. We verified that a single peak was obtained for each product using the Roche software. Amplimers were all normalized to corresponding L32 values. Data analysis was carried out using the comparative Ct method: in real-time each replicate average genes C_{T} was normalized to the average C_{T} of L32 by subtracting the average C_{T} of L32 from each replicate to give the ACT. Results are expressed as Log^{-2 ΔΔCT} with ΔΔCT equivalent to the ΔC_{T} of the genes in PC3 (clone, XCT), tumors or treated MC3T3-E1 subtracting to the AC_{T} of the endogenous control (empty vectors control for clones or tumors, DMSO for the XCT experiment and empty vectors control conditioned media treated MC3T3-E1 respectively).

### Statistical Analysis

Pair wise comparisons were carried out by conducting a non parametric Mann-Whitney U test for in vivo studies. Data were analyzed statistically by one way analysis of variance (ANOVA) followed by post hoc t-tests to assess the differences between groups for in vitro studies. Results of p<0.05 were considered significant: *=p<0.05; **=p<0.001; ***=p<0.0001

### Results:

ERRα mRNA and Protein Expression in Human Primary Prostate Tumors and Bone Metastases

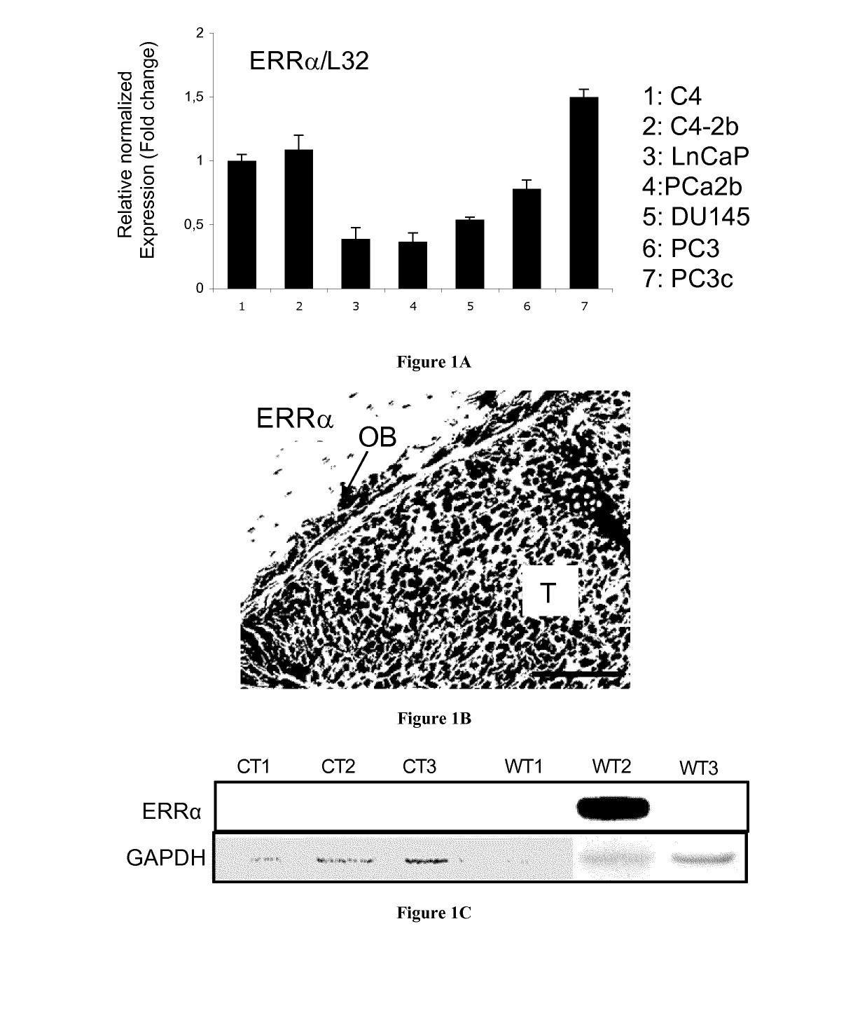

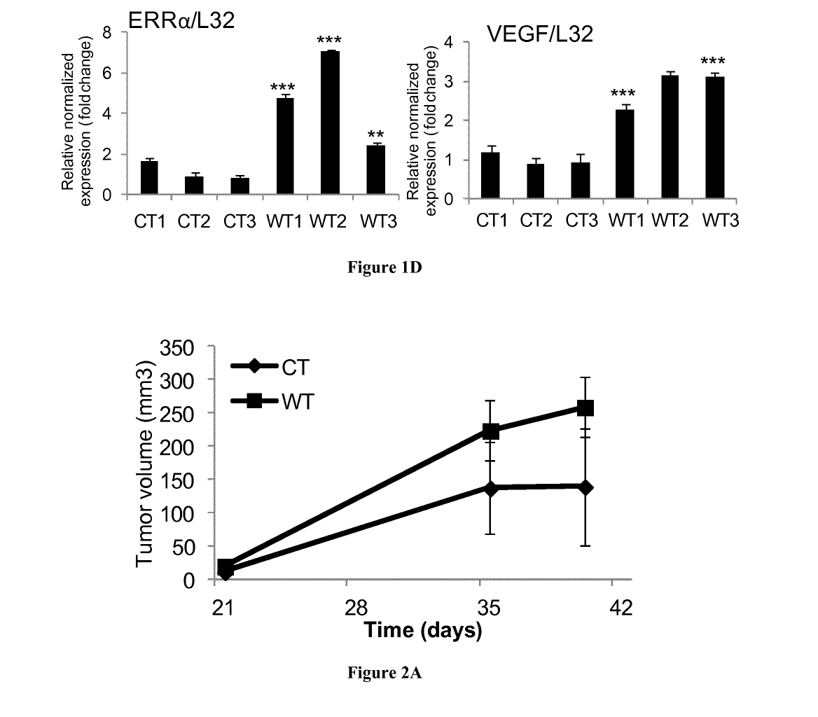

We analyzed ERRα mRNA expression by real-time RT-PCR in human prostate cancer cell lines including AR-positive cell lines C4, C4-2b, LnCaP, PCa2b and AR-negative cell lines DU145, and PC3. Real-time RT-PCR revealed that ERRα mRNA levels were almost similar in C4, C4-2b, and PC3 whereas lower ERRα mRNA levels were found in LnCaP and PCa2b cells (FIG. 1A). To assess whether ERRα is involved in bone metastases formation, we used PC3 cells model, known for their high efficiency to form pure osteolytic lesions in bone in vivo (Akech et al. 2010). ERRα protein was also seen in the nucleus and cytoplasm of PC3 cells in situ in bone metastases from legs of animals, 6 weeks after intra-tibial cell inoculation (FIG. 1B). As we previously shown ERRα was also detected in osteoblasts (FIG. 1B) (Bonnelye et al. 2001).

To establish a functional role for ERRα in bone metastases development, we next stably transfected PC3 cells with a construct encoding for a full-length (wild type; WT) ERRα (FIG. 1C). Alternatively, PC3 cells were stably transfected with the vector alone, which was used here as a control (CT). Three independent PC3-ERRα-WT, and three PC3-CT clones were obtained, named WT1-3 and CT1-3 respectively. As judged by real-time PCR, total ERRα mRNA expression was increased by 2 to 7-fold in WT clones, when compared to CT-1-3 clones (FIG. 1D). Western blotting detected a band of approximately 50 kD for ERRα protein in CT 1-3 and WT1-3 cells (FIG. 1C). Compared to CT 1-3 cells, ERRα protein expression was increased in WT1-3 cells (FIG. 1C). mRNA expression levels of the ERRα target gene VEGF were statistically significantly increased in WT1-3 cells compared to CT-1-3 cells (FIG. 1D).

### ERRα Stimulates Tumor Growth In Vivo

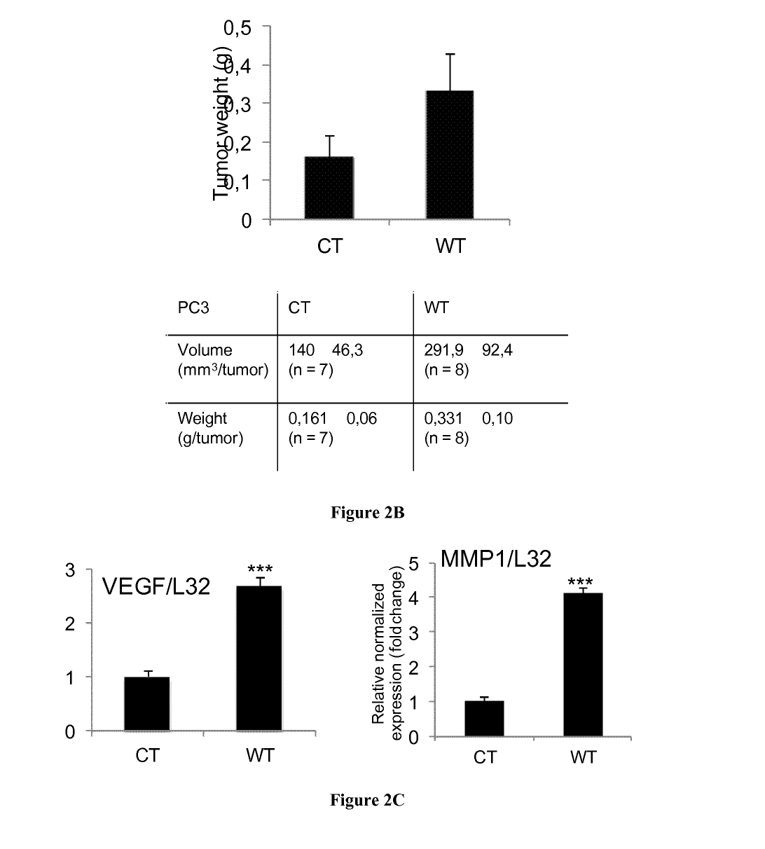

ERRα has been implicated in tumor progression. To assess the involvement of ERRα in primary prostate tumor progression in PC3 cells, CT (pool of CT-1, -2 and -3 clones), and WT (pool of WT1, -2 and -3 clones) cells were inoculated subcutaneously in SCID male mice and tumors were induced. Tumor progression follow-up from day 21 to day 42 revealed a greater tumor progression in WT tumor-bearing animals compared to that observed with CT (FIG. 2A). Tumor weight/size at day 42 (FIG. 2B) correlated well the tumor progression observed between day 21 to day 42 (FIG. 2A, B). Interestingly, and similarly to breast tumor, there was a positive association between high levels of ERRα and VEGF and MMP1 in PC3 prostate tumors (FIG. 2C) (Dwyer et al. 2010; Fradet et al. 2011; Stein R A et al. 2009). Taken together, and similarly to breast cancer, our results strongly suggest that ERRα promoted tumor growth, mainly through the stimulation of angiogenesis and invasion in prostate cancer.

### ERRα Expression in Prostate Cancer Cells Increase Their Ability to Induce Osteolytic Lesions but Also to Induce New Bone Formation In Vivo

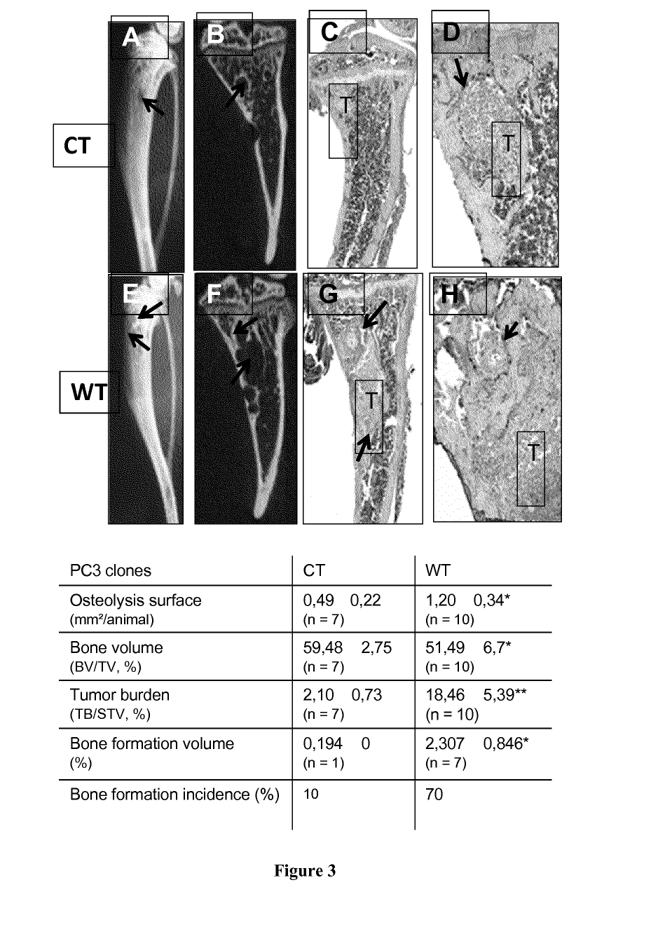

To assess the involvement of ERRα in bone metastases formation, CT (pool of CT- 1, -2 and -3 clones), and WT (pool of WT1, -2 and -3 clones) cells were inoculated by intra-tibial injection into male SCID mice. Three weeks after tumor cell injection, radiographic analysis revealed that animals bearing WT tumors had an increase of 40% in osteolytic lesions formation compared than those of mice bearing CT tumors (CT:0.49±0.22 versus WT:1.20±0.34) (FIG. 3A, D; G). The stimulatory effect of ERRα on prostate cancer-induced bone destruction was confirmed using three-dimensional microCT reconstruction (bone volume (BV/TV, %)) (FIG. 3B, E, G), histology (FIG. 3C, F) and histomorphometric analyses of tibiae as BV/TV was decreased and skeletal tumor burden (TB/STV) increased (FIG. 3G). Very interestingly, there was an incidence of 70% in animals bearing WT metastases that was presenting new bone formation compared than those of mice bearing CT metastases that only developed lytic lesions (FIG. 3G). That result was not expected in PC3 model that is known to induce only pure osteolytic lesions in vivo suggesting that over-expressing ERRα in prostate cancer cells may be associated to mechanisms mediating mixed metastatic lesions in vivo. Taken together, our results indicated that over-expression of ERRα in prostate cancer cells stimulate process of bone remodeling associating resorption and new bone formation.

### Regulation of Osteoclast and Osteoblast Formation and Markers by ERRα Expressing PC3 Cells

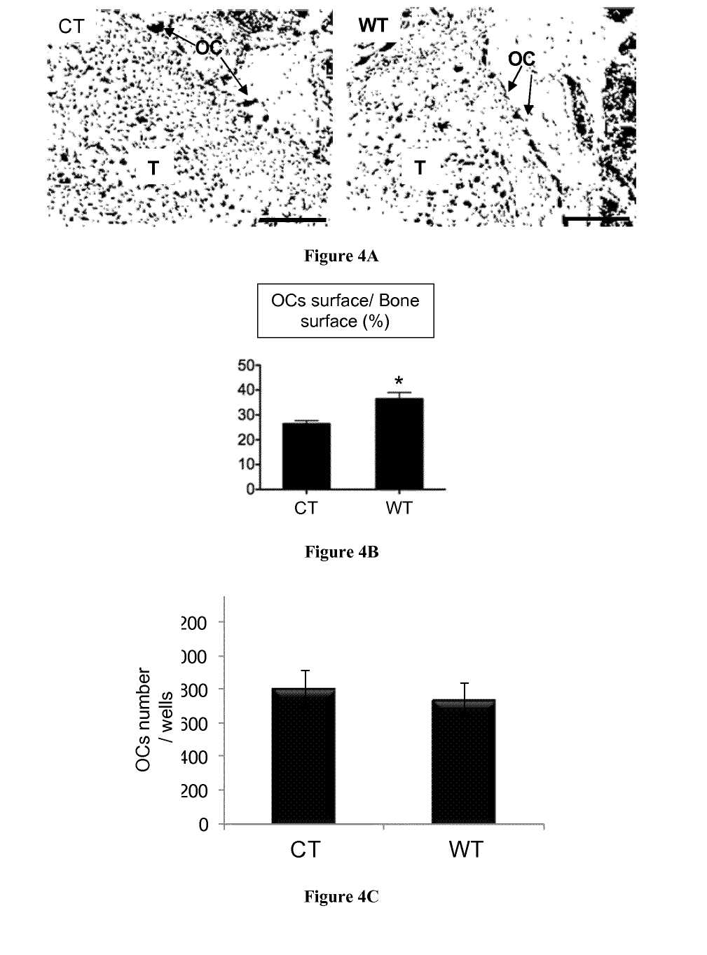

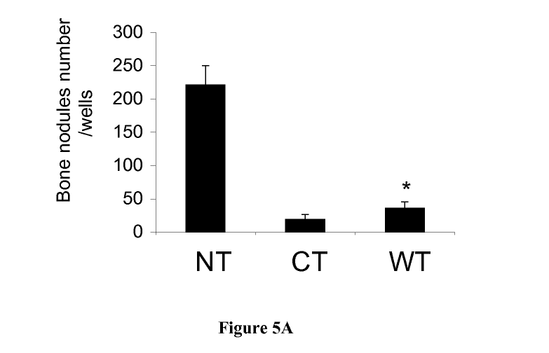

Given the effect of tumor-derived ERRα on the development of osteolytic lesions and new bone formation, we next asked whether modulation of ERRα in prostate cancer cells could alter the number of osteoclasts (OCs), the bone resorbing cells. Tartrate-resistant acid phosphatase (TRAP) staining of tibial sections of metastatic legs from animals bearing WT and CT tumors showed only an increase TRAP-positive OC surface (Oc.S/BS) at the bone/tumor cell interface in WT, when compared to CT tumors (FIG. 4A-B). In parallel, the treatment of primary mouse bone marrow cell cultures with RANKL and macrophage colony-stimulating factors (M-CSF) together with the conditioned medium extracted from WT cells show no effect on the formation of TRAP-positive multinucleated OCs compared to that observed with the conditioned medium of CT cells (FIG. 4C) suggesting that there is a direct impact on OC by cancerous cells modified by ERRα.

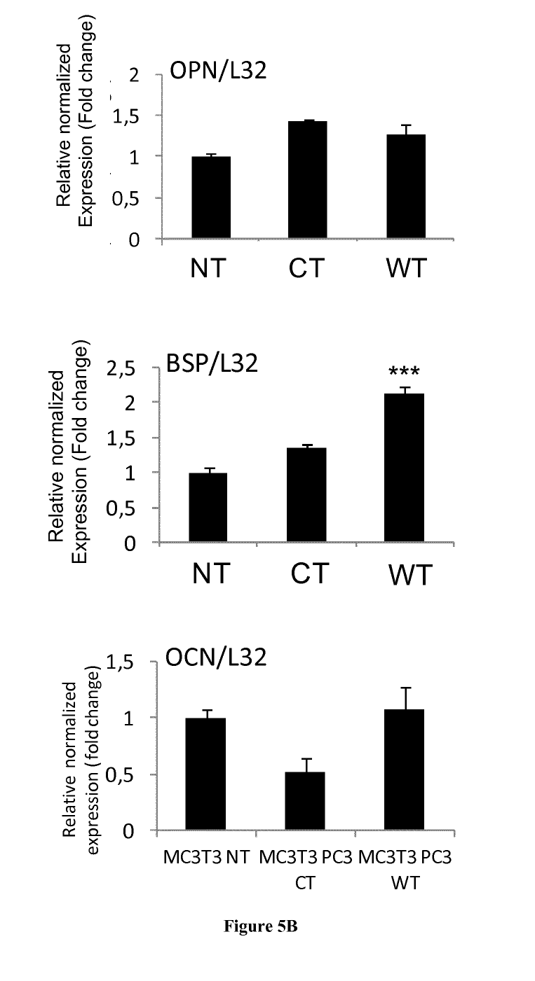

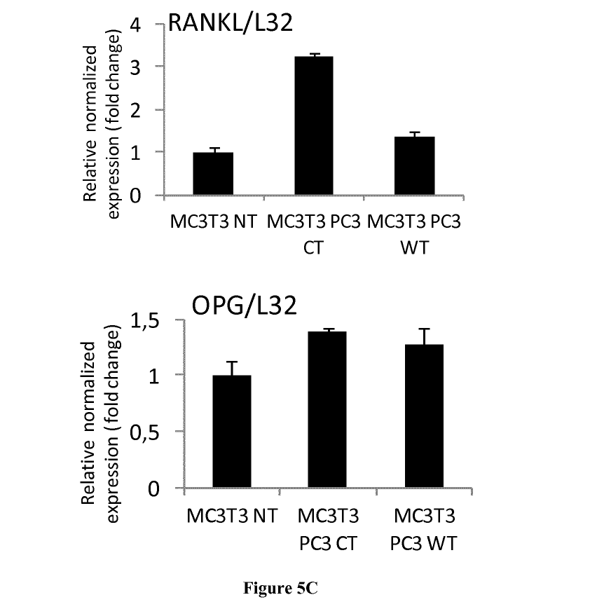

We next asked whether modulation of ERRα activity in PC3 cells could alter the number of osteoblasts (OBs), the bone forming cells. Even though fewer mineralized bone nodules were formed with conditioned media from PC3 transfectants irrespective of the ERRα status of transfected PC3 cells (FIG. 5A; NT versus CT and WT), primary mouse calvaria OBs cultured with conditioned media of WT formed more mineralized bone nodules compared with the conditioned medium of CT cells (FIG. 5A; CT versus WT). Concomitantly, expression of bone sialoprotein (BSP) and osteocalcin (OCN), a mature OB marker, was markedly increased (FIG. 5 B; CT versus WT), in MC3T3-E1 osteoblastic cells when cells were treated for 6 days with WT conditioned media compared CT. Expression of osteopontin (OPN), an early OB marker, was not regulated (FIG. 5 B; CT versus WT). In addition, the increase in ERRα activity led to an decrease of RANKL while OPG was not affected (FIG. 5 C; CT versus WT). Taken together, our results indicated that over-expression of ERRα in prostate cancer cells can stimulate osteoblast formation which is associated to an increase in BSP, OCN and a decrease in RANKL expression. OCN that is the most abundant non-collagenous protein of the bone extracellular matrix, can enter the systemic circulation when undercarboxylated (ucOCN) and is believed to act as a hormone to alter insulin production by pancreatic β-cells and global glucose homeostasis (Ferron et al. 2011; Lee et al. 2007). Interestingly, insulin itself regulates bone metabolism stimulating osteoblastogenesis and OCN expression (Ferron et al. 2010; Fulzele et al. 2010) suggesting that in vivo PC3 cells over-expressing ERRα may stimulate directly and indirectly via the OCN pathway, OB formation contributing to the unexpected new bone formation in animals bearing WT tumor cells. On the otherside, our data suggest that in vivo PC3 cells over-expressing ERRα may indirectly inhibit OC via inhibited RANKL production in OB. It is also worth to mention that OCN can also regulate bone metabolism by increasing bone resorption as OCN can attenuate expression of osteoprotegerin (OPG) in OB (Fulzele et al. 2010).

### Regulation of Molecules Implicated in Bone Remodeling by ERRα in Prostate Cancer Cells.

Prostate cancer cells metastatic to bone (but not to soft tissues) mimic some of the characters of normal cells present in the host organ by expressing osteoblastic and/or osteoclastic genes, thereby helping these cancer cells to adapt and thrive in the bone marrow microenvironment (Bellahcene, et al. 2007). To determine whether modulation of ERRα levels in prostate cancer cells alters OC markers and/or molecules implicated in bone resorption, we quantified and found that Runx2, (a master gene in OB differentiation that is associated to prostate cancer-induced bone resorption), cathepsin K (CK) (protease that can degrade bone matrix and increase osteolytic lesions in breast cancer derived bone metastases), monocyte chemotactic protein-1 (MCP-1) (chemokine that can stimulate OC formation and mediates prostate cancer-induced bone resorption), TGFβ1 (growth factor that can stimulate OC formation and osteolytic lesion formation) were up-regulated in PC3-WT primary tumors in vivo compared PC3-CT tumors (FIG. 6A) (Akech et al. 2010; Komori. 2002; Le Gall et al. 2007; Lu et al. 2007; Weilbaecher et al. 2011). Contrasting the positive regulation of these pro-osteoclastic factors, we confirm, in prostate cancer cells, our previous data in breast cancer cells concerning the regulation of OPG (inhibitor of OC (RANKL antagonist)) by ERRα (Fradet et al. 2011) (FIG. 6A). The regulation of these factors associated to the increased of OCN expression will eventually contributing to the fact that all together PC3 over expressing ERRα had a global impact on OC in vivo despite the decreased of RANKL expression observed in OB induced by WT conditioned media.

To determine whether modulation of ERRα levels in prostate cancer cells alters OB markers and/or molecules implicated in bone formation, we quantified and found that endothelin-1 (ET1) (a mitogenic factor for OB involved in osteoblastic lesion formation), and Wnt pathway factors Wnt3a, Wnt5a, Wnt10b and Wnt11 (that had also been shown regulated by ERRα in breast cancer cells) were up-regulated in PC3-WT primary tumors in vivo compared PC3-CT tumors (FIG. 6B) (Dwyer et al. 2010). It's worth to mention that TGFβ1 is also known to be able to recruit mesenchymal stem cells and direct them along the osteoblast lineage contributing to increase OB formation. Contrasting the positive regulation of these pro-osteoblastic factors, we found that DKK1 (Wnt antagonist), is also regulated in prostate cancer cells over-expressing ERRα (FIG. 6B). In breast cancer models, it has been shown that ERRα associated with β-catenin directly regulates Wnt11 expression, documenting a crosstalk between ERRα and the Wnt pathway (Dwyer et al, 2010). ERRα expression enhanced β-catenin-dependent transcription from a T cell factor/Lymphoid enhancer-binding factor-1 (TCF/LEF-1) reporter and ERRα was shown to directly interact with 13-catenin and LEF-1 (Dwyer et al, 2010). Wnt/β-catenin signaling is required for OB differentiation and plays a key role in deciding the fate of mesenchymal stem cells during development and in mature bone (Hall et al, 2006). The latter is supported by the fact that overexpression of ERRα in C3H10T1/2 cells markedly induces Wnt11 expression and other Wnt pathway expression changes (Auld et al, 2012). Taken together this results suggest that modulation of ERRα in prostate cancer cells can stimulate OB correlating our data obtained in vitro in primary osteoblasts and MC3T3-E1 and in vivo with the new bone formation observed in bone metastases induced by PC3-WT cells while only osteolytic lesions were observed in bone metastases induced by PC3-CT (FIG. 3).

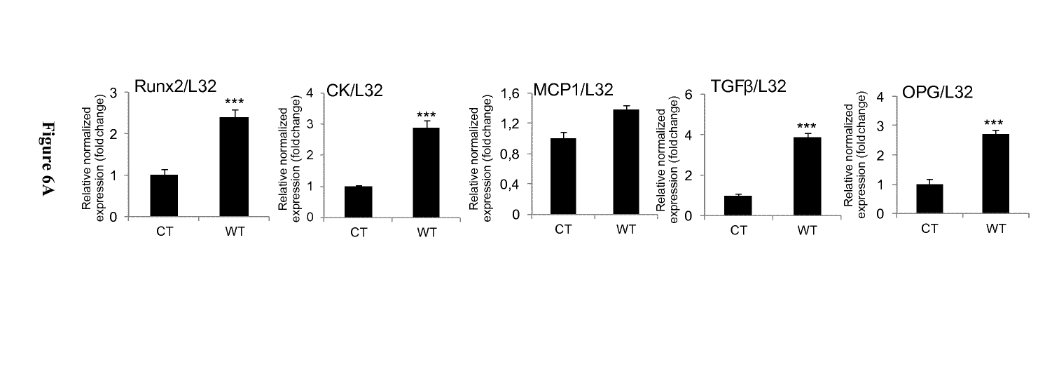

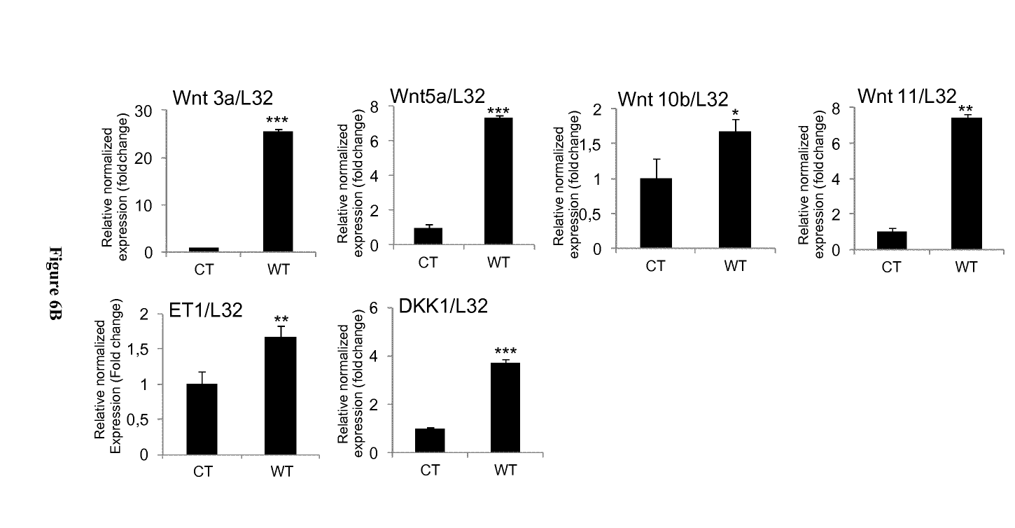

We then confirmed, in vitro, by real-time PCR the regulation of Runx2, CK, MCP-1, TGFβ, OPG, Wnt3a, Wnt10b, ET1 and DKK1 in each clones independently (FIG. 7A, B). We have to acknowledge the fact that few factors were regulated in WT3 which is probably due to the fact that ERRα mRNA is only induce 2× compared WT1 (5×) and WT2 (7×). Moreover Wnt5a and Wnt11 were only regulated in WT1 cells (FIG. 7B) suggesting that other mechanisms may occur in vivo. FGF9 known to induce mixed lesions formation by prostate cancer cells was also up-regulated in PC3 clones over-expressing ERRα. Interestingly, FGF9had already been described having in his promoter an ERRα binging site (Li et al. 2008; Tremblay et al. 2010).

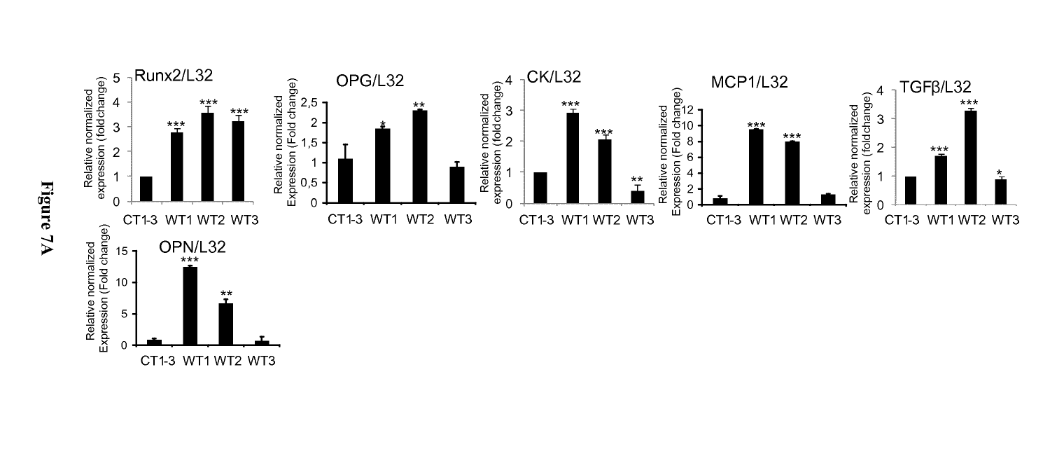

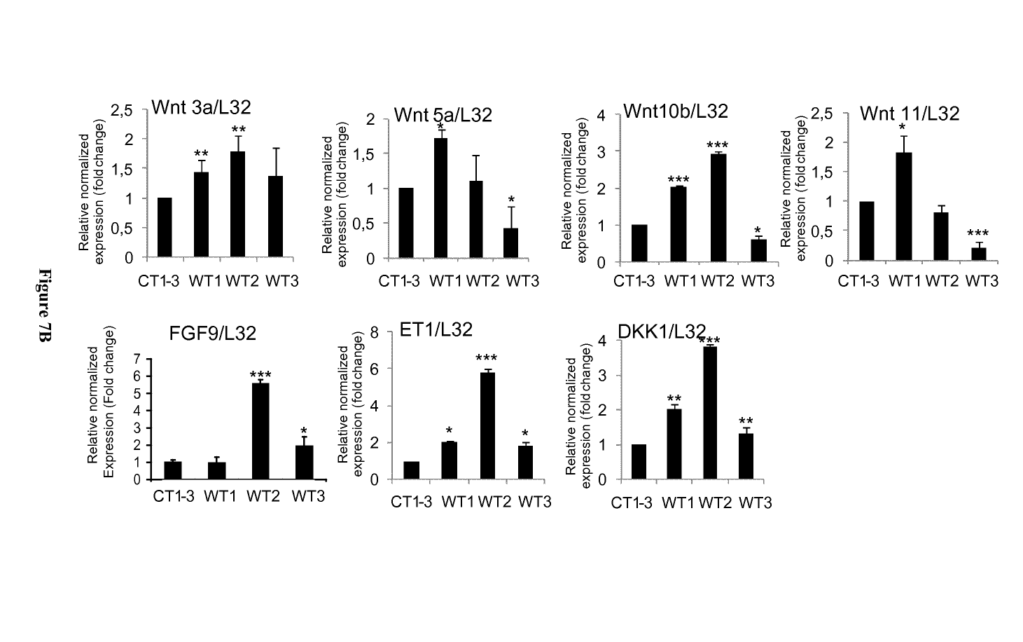

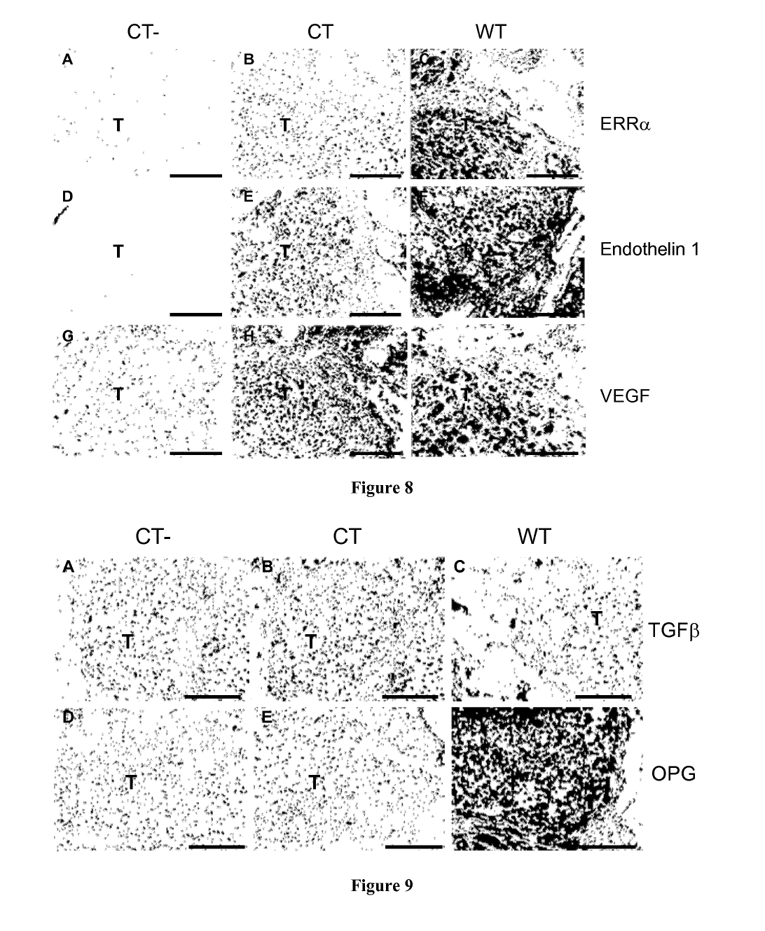

We therefore examined ERRα, ET1, VEGF, TGFβ1 and OPG expression in bone metastatic specimens from animals bearing CT or WT tumors, using immunohistochemistry (FIG. 8 and 9). As expected ET1 (FIG. 8E-F), VEGF (FIG. 8H-I), TGFβ active form (FIG. 9 B-C) and OPG (FIG. 9E-F) protein expression was highly increased in bone metastastatic cells over-expressing ERRα WT compared CT (FIG. 8B-C), even though TGFβ stimulation was less striking.

### EXAMPLE 2

### ERRα and Metastatic Nich: Implication of the Wnt/Periostin Pathway

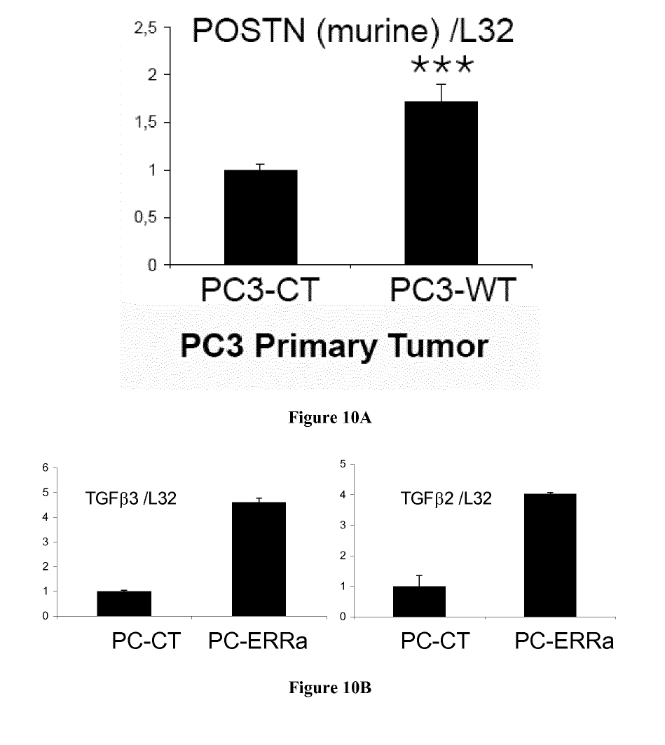

We recently found that ERRalpha is positively associated with the expression of the periostin (POSTN) in cancer-associated fibroblasts (CAFs) of prostate cancer (immunohistochemistry and RT-PCR). POSTN is increased in tumor and is crucial to promote tumor metastases, invasion and angiogenesis (Wang and Ouyang, 2012, cancer stem cell; 10:111-112). Indeed, PC3 cells over expressing ERRalpha induce POSTN expression in stromal cells which let us to suggest that ERRalpha, in tumor cells, may impact tumor environment by for instance modulating level of stromal factors expression (POSTN) within tumor environment and play a key role in the paracrine signaling from cancer cells to CAFs (FIG. 10). Finally TGFbeta2 and 3 from tumor cells that are known to stimulate stromal POSTN were found up-regulated in PC3 cells over-expressing ERRalpha (Malanchi et al, 2011, Nature, 481(7379-85)). Moreover, these observation combined with the up-regulation of several Wnt members (Wnt3a, 5a, 10b, 11) by ERRalpha in the PC3-ERRα cells compare control PC3 cells suggest that ERRalpha may be also implicated in the early phase of metastatic colonization through the WNT/POSTN pathway (Malanchi et al, 2011, Nature, 481(7379-85)) and therefore participate to the establishment of the metastatic niche.

Based on these data, we can also speculate that ERRα may also regulate angiogenesis not only through the direct regulation of VEGF but also through the regulation of POSTN and the perivascular niche.

### EXAMPLE 3

### ERRα and Metastatic Nich: Implication of the Adiponectin Pathway

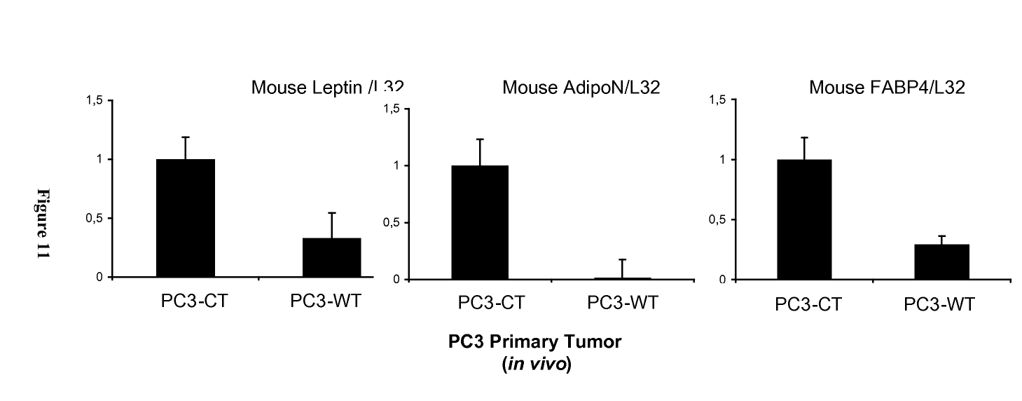

We recently found, by using RT-PCR and host primers (mouse) that in vivo in tumor generated by over expressing human ERRα-PC3 cells, host adipocytes markers such as mouse adiponectin, leptin and FABP4 were dramatically decreased (FIG. 11) suggesting that ERRα in tumor cells can modify tumoral environment by inhibiting adipose tissues-derived stromal cells. It worth mentioning that adiponectin and leptin are both adipokines that are inhibitors of osteoclastogenesis which may also explain the fact that bone lesions are increased when PC3-overexpressing ERRα cells are injected compared control PC3 cells.

### EXAMPLE 4

### ERRα and Prostate Cancer Cells Osteomimetism

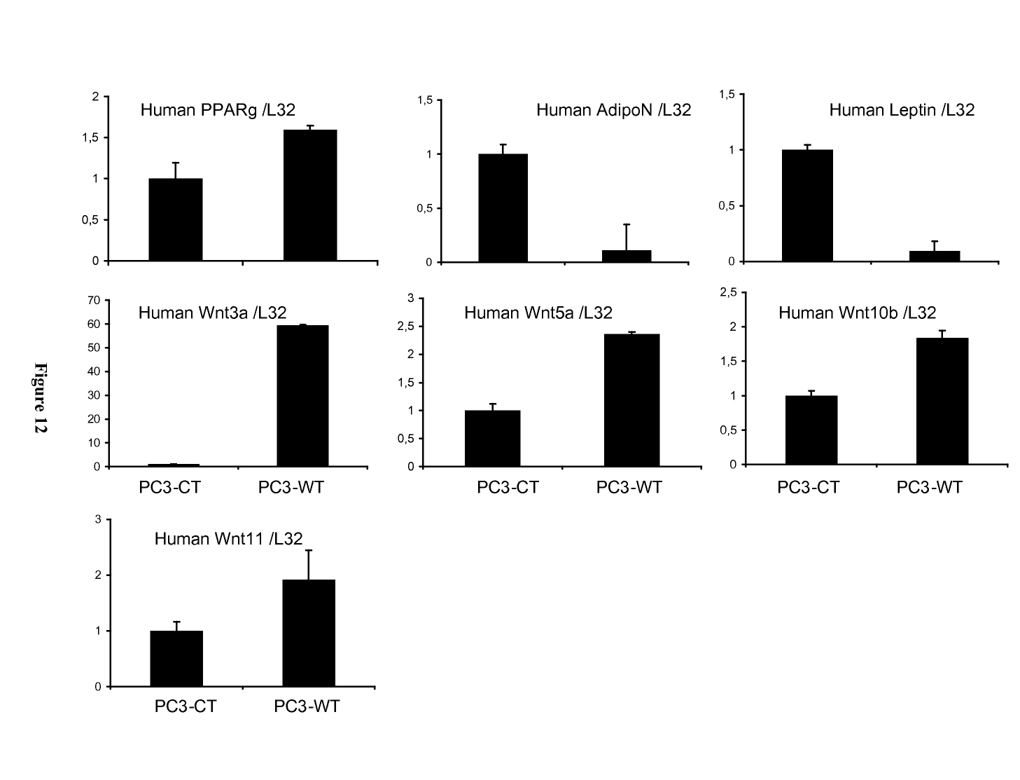

By looking at human adipocyte markers adiponectin and leptin in vivo, we also found that both adipokines expression was also down-regulated in tumor cells (FIG. 12). Combined with the up-regulation of the osteoblastic factors Wnt, these data suggest that ERRα may tigger tumor cells toward osteoblast phenotype by inhibiting adipokines and stimulating osteoblasts factors which may contribute to stimulate osteolysis.

### EXAMPLE 5

### ERRα and Cancer Stem Cells

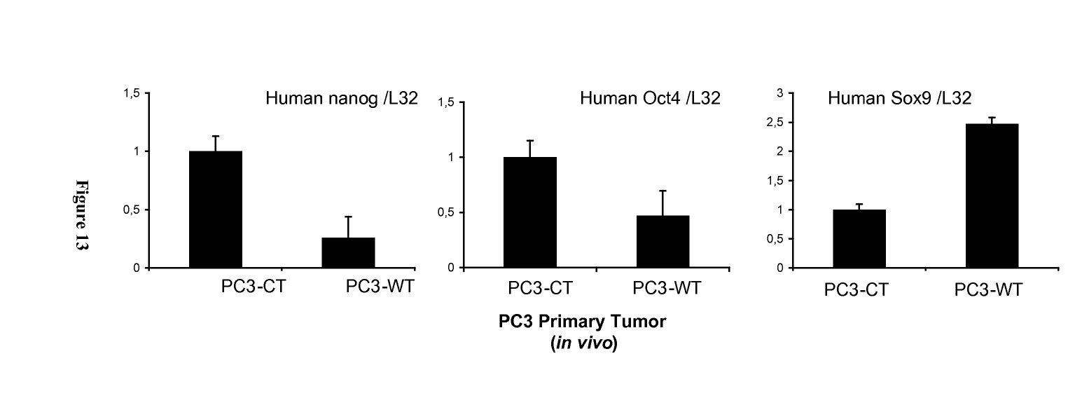

Cancer stem cells (CSC), like normal stem cells, may exist at niches in bone marrow and it is necessary to disrupt cancer stem-cell niche interactions or eradicate cancer stem cells to achieve a better cure for metastatic breast or prostate cancer. POSTN might affect the maintenance and expansion of CSC during metastatic colonization through Wnt1 and Wnt3a (Malanchi et al, 2011, Nature, 481(7379-85) and extensives investigations also identify general Wnts signal in the regulation of CSC (Curtin and Lorenzi, oncotarget, 2010; 1(7):552-66) suggesting that ERRα through its regulation of the POSTN/Wnt pathway may be implicated in cancer cells self-renewal and CSC expansion. Interestingly and at the inverse, we found that in PC3 cells over expressing ERRα, Nanog and Oct4 (two stem cells markers) expression was decreased while Sox9 is increased suggesting that tumoral ERRα may limits cells self-renewal and CSC expansion (FIG. 13).

### EXAMPLE 6

### Confirmation in Clinic

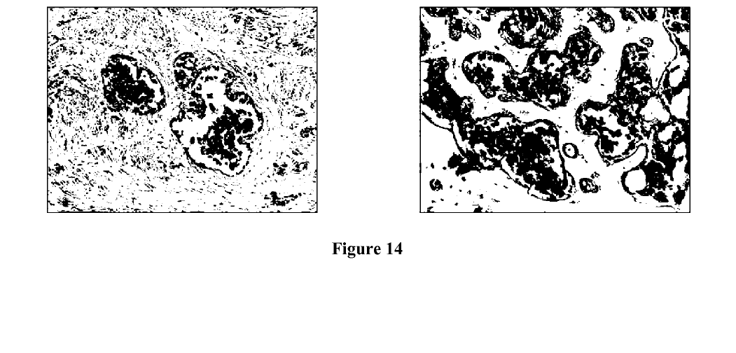

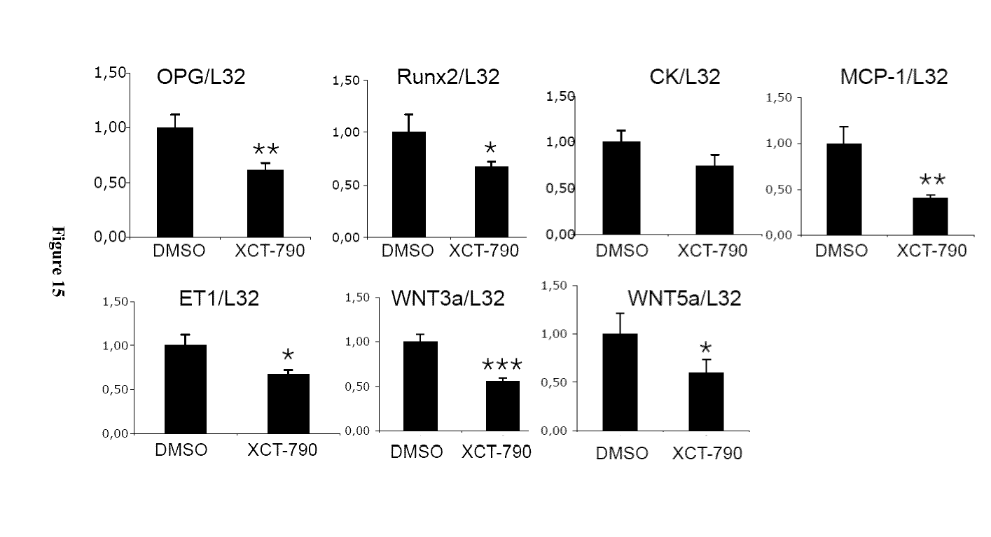

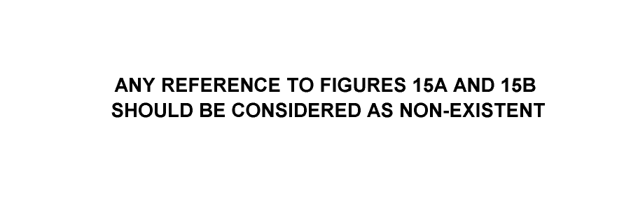

We confirmed by immuhistochemistry that ERRα expression in prostate cancer biopsy obtained from patients was also maintained in associated bone metastases (FIG. 14).

### EXAMPLE 7

### Regulation of Several Factors by the ERRalpha Inverse Agonist XCT-790 in PC3 Cells

To confirm the regulation of the factors identified in PC3 over-expressing ERRα, we treat PC3 cells by the inverse agonist XCT790 (5 10⁻⁷M) for 24 hours. XCT790 is able to block the interaction between ERRα and his co-activators PGC1a and b that will have as a consequence the inhibition of ERRα transcriptional activity. We found that the osteolytic factors Runx2, and MCP1 but no CK were found statistically down-regulated after XCT treatment such as OPG. Moreover the osteoforming factors ET1, Wnt3a and Wnt5a were also identified as direct ERRα/PGC1 target genes.

### REFERENCES

Throughout this application, various references describe the state of the art to which this invention pertains. The disclosures of these references are hereby incorporated by reference into the present disclosure.
Akech J W J, Bedard K, van der Deen M, Hussain S, Guise T A, van Wijnen and A J S J, Languino L R, Altieri D C, Pratap J, Keller E, Stein G S, Lian J B. Runx2 association with progression of prostate cancer in patients: mechanisms mediating bone osteolysis and osteoblastic metastatic lesions. 2010; Oncogene 29 (6) 811-821.
Ariazi E A, Clark G M and Mertz J E. Estrogen-related receptor alpha and estrogen-related receptor gamma associate with unfavorable and favorable biomarkers, respectively, in human breast cancer. 2002; Cancer Res 62 (22) 6510-6518.
Auld K L, Berasi S P, Liu Y, Cain M, Zhang Y, Huard C, Fukayama S, Zhang J, Choe S, Zhong W, Bhat B M, Bhat R A, Brown E L, Martinez R V. Estrogen-related receptor a regulates osteoblast differentiation via Wnt/β-catenin signaling. 2012 JEM 48 (2) 177-91
Bellahcene A, Bachelier R, Detry C, Lidereau R, Clezardin P and Castronovo V. Transcriptome analysis reveals an osteoblast-like phenotype for human osteotropic breast cancer cells. 2007; Breast Cancer Res Treat 101 (2) 135-148.
Bellahcene A, Castronovo V, Ogbureke K U, Fisher L W and Fedarko N S. Small integrin-binding ligand N-linked glycoproteins (SIBLINGs): multifunctional proteins in cancer. 2008; Nat Rev Cancer 8 (3) 212-226.
Benoit G, Cooney A, Giguere V, Ingraham H, Lazar M, Muscat G, Perlmann T, Renaud J P, Schwabe J, Sladek F, Tsai, M. J. and Laudet, V. International Union of Pharmacology. LXVI. Orphan nuclear receptors. 2006; Pharmacol Rev 58 (4) 798-836.
Bonnelye E, Merdad L, Kung V and Aubin J E. The orphan nuclear estrogen receptor-related receptor (ERRa) is expressed throughout osteoblast differentiation and regulates bone formation in vitro. 2001; J Cell Biol 153 (5) 971-983.
Bonnelye E, Vanacker J M, Spruyt N, Alric S, Fournier B, Desbiens X and Laudet V. Expression of the estrogen-related receptor 1 (ERR-1) orphan receptor during mouse development. 1997; Mech Dev 65 (1) 71-85.
Bonnelye E, Zirngibl R A, Jurdic P and Aubin J E. The Orphan Nuclear Estrogen Receptor-Related Receptor-{alpha} Regulates Cartilage Formation in Vitro: Implication of Sox9. 2007; Endocrinology 148 (3) 1195-1205.
Bonnelye E. Saltel, F. and Jurdic, P. Dual effect of strontium ranelate: stimulation of osteoblast differentiation and inhibition of osteoclast formation and resorption in vitro. 2008; Bone 42 (1) 129-138.
Bonnelye E, F. Chabadel, A. Zirngibl, R A. Aubin, J E. and Jurdic, P. Involvement of the orphan nuclear estrogen receptor-related receptor ERRa in osteoclast adhesion and transmigration. 2010; J Mol Endocrinol 45 (6) 365-377.
Boyle W J, Simonet W S and Lacey D L. Osteoclast differentiation and activation. 2003; Nature 423 337-342.
Busch B B S W J, Martin R, Ordentlich P, Zhou S, Sapp D W, Horlick R A, and Mohan R. Identification of a selective inverse agonist for the orphan nuclear receptor estrogen-related receptor alpha. 2004; J Med Chem 47 (23) 5593-5596.
Cavallini A, Notarnicola M, Giannini R, Montemurro S, Lorusso D, Visconti A, Minervini F and Caruso M G. Oestrogen receptor-related receptor alpha (ERRalpha) and oestrogen receptors (ERalpha and ERbeta) exhibit different gene expression in human colorectal tumour progression. 2005; Eur J Cancer 41 (10)1487-1494.
Cheung C P, Yu S, Wong K B, Chan L W, Lai F M, Wang X, Suetsugi M, Chen S and Chan F L. Expression and functional study of estrogen receptor-related receptors in human prostatic cells and tissues. 2005; J Clin Endocrinol Metab 90 (3) 1830-1844.
Chisamore M J, Wilkinson H A, Flores O, Chen J D 2009 Estrogen-related receptor-alpha antagonist inhibits both estrogen receptor-positive and estrogen receptor-negative breast tumor growth in mouse xenografts. Mol Cancer Ther 8(3):672-81
Delhon I, Gutzwiller S, Morvan F, Rangwala S, Wyder L, Evans G, Studer A, Kneissel M and Fournier B. Absence of Estrogen Receptor Related Alpha Increases Osteoblastic Differentiation and Cancellous Bone Mineral Density. 2009; Endocrinology 150 (10) 4463-4472.
Dwyer M A J J, Wade H E, Eaton M L, Kunder R S, Kazmin D, Chang C Y and McDonnell D P. WNT11 expression is induced by estrogen-related receptor alpha and beta-catenin and acts in an autocrine manner to increase cancer cell migration. 2010; Cancer Res 70 (22) 9298-308.
Ferron M M M, Levine R L, Ducy P and Karsenty G. Intermittent injections of osteocalcin improve glucose metabolism and prevent type 2 diabetes in mice. 2011; Bone 50 (2) 568-575.
Ferron M W J, Yoshizawa T, Del Fattore A, DePinho R A, Teti A, Ducy P, and Karsenty G. Insulin signaling in osteoblasts integrates bone remodeling and energy metabolism. 2010; Cell 142 (2) 296-308.
Fiori J L, Sanghvi M, O'Connell M P, Krzysik-Walker S M, Moaddel R, Bernier M. The cannabinoid receptor inverse agonist AM251 regulates the expression of the EGF receptor and its ligands via destabilization of oestrogen-related receptor a protein. Br J Pharmacol. 2011 October; 164(3):1026-40.
Fradet A S H, Bouazza L, Goehrig D, Dépalle B, Bellahcène A, Castronovo V F H, Descotes F, Aubin J E, Clézardin P and Bonnelye E. Dual function of ERRa in breast and bone metastases formation: implication of VEGF and osteoprotegerin.2011; Cancer Res 71 (17) 5728-5738.
Fujimura T, Takahashi S, Urano T, Kumagai J, Ogushi T, Horie-Inoue K, Ouchi Y, Kitamura T, Muramatsu M and Inoue S. Increased expression of estrogen-related receptor alpha (ERRalpha) is a negative prognostic predictor in human prostate cancer. 2007; Int J Cancer 120 (11) 2325-2330.
Fulzele K R R, DiGirolamo D J, Cao X, Wan C, Chen D, Faugere M C, Aja S, Hussain M A B J, and Clemens T L. Insulin receptor signaling in osteoblasts regulates postnatal bone acquisition and body composition. 2010; Cell 142 (2) 309-319.
Gao M, Sun P, Wang J, Zhao D and Wei L. Expression of estrogen receptor-related receptor isoforms and clinical significance in endometrial adenocarcinoma. 2006; Int J Gynecol Cancer 16 (2) 827-833.
Giguere V, Yang N, Segui P and Evans R M. Identification of a new class of steroid hormone receptors. 1988; Nature 331 (6151) 91-94.
Hall C L K S, MacDougald O A and Keller E T. Role of Wnts in prostate cancerbone metastases. 2006; J Cell Biochem 97 (4) 661-72.
Huss J M, Imahashi K, Dufour C R, Weinheimer C J, Courtois M, Kovacs A, Giguere V, Murphy E and Kelly D P. The nuclear receptor ERRalpha is required for the bioenergetic and functional adaptation to cardiac pressure overload. 2007; Cell Metab 6 (1) 25-37.
Kallen J, Schlaeppi J M, Bitsch F, Filipuzzi I, Schilb A, Riou V, Graham A, Strauss A, Geiser M and Fournier B. Evidence for ligand-independent transcriptional activation of the human estrogen-related receptor alpha (ERRalpha): crystal structure of ERRalpha ligand binding domain in complex with peroxisome proliferator-activated receptor coactivator-1alpha. 2004; J Biol Chem 279 (47) 49330-49337.
Komori T. Runx2, a multifunctional transcription factor in skeletal development. 2002; J Cell Biochem 87 (1) 1-8.
Le Gall C B A, Bonnelye E, Gasser J A, Castronovo V, Green J, Zimmermann J, Clézardin P. A cathepsin K inhibitor reduces breast cancer induced osteolysis and skeletal tumor burden. 2007; Cancer Res 67 (20) 9894-9902.
Lee N K S H, Hinoi E, Ferron M, Ahn J D, Confavreux C, Dacquin R, Mee P J, & McKee M D J D, Zhang Z, Kim J K, Mauvais-Jarvis F, Ducy P and Karsenty G. 2007; Cell 130 (3) 456-469.
Li Z G M P, Yang J, Starbuck M W, Zurita A J, Liu J, Sikes C, Multani A S, Efstathiou E L A, Wang J, Fanning T V, Prieto V G, Kundra V, Vazquez E S, Troncoso P R A, Logothetis C J, Lin S H, Maity S and Navone N M. Androgen receptor-negative human prostate cancer cells induce osteogenesis in mice through FGF9-mediated mechanisms. 2008; J Clin Invest. 118 (8) 2697-2710.
Logothetis C J and Lin S H. Osteoblasts in prostate cancer metastasis to bone. 2005; Nat Rev Cancer 5 (1) 21-28.
Lu Y C Z, Xiao G, Keller E T, Mizokami A, Yao Z, Roodman G D and Zhang J. Monocyte chemotactic protein-1 mediates prostate cancer-induced bone resorption. 2007; Cancer Res 67 (8) 3646-3653.
Luo J, Sladek R, Carrier J, Bader J A, Richard D and Giguere V. Reduced fat mass in mice lacking orphan nuclear receptor estrogen-related receptor alpha. 2003; Mol Cell Biol 23 (22) 7947-7956.
Patch R J, Searle L L, Kim A J, De D, Zhu X, Askari H B, O'Neill J C, Abad M C, Rentzeperis D, Liu J, Kemmerer M, Lin L, Kasturi J, Geisler J G, Lenhard J M, Player M R, Gaul M D 2011 Identification of diaryl ether-based ligands for estrogen-related receptor alpha as potential antidiabetic agents. J Med Chem 54(3):788-808
Rajalin A M P H, Pollock H and Aarnisalo P. ERRalpha regulates osteoblastic and adipogenic differentiation of mouse bone marrow mesenchymal stem cells. 2010; Biochem Biophys Res Commun. 396 (2) 477-482.
Roodman G D. Mechanisms of bone metastasis. 2004; N Engl J Med 350 (16) 1655-1664.
Sladek R, Bader J A and Giguere V. The orphan nuclear receptor estrogen-related receptor alpha is a transcriptional regulator of the human medium-chain acyl coenzyme A dehydrogenase gene. 1997; Mol Cell Riot 17 (9) 5400-5409.
Stein R A, Gaillard S, McDonnell D P. Estrogen-related receptor alpha induces the expression of vascular endothelial growth factor in breast cancer cells. 2009; J Steroid Biochem Mol Biol 114 (1) 106-112.
Stein R A and McDonnell D P. Estrogen-related receptor {alpha} as a therapeutic target in cancer. 2006; Endocr Relat Cancer 13 Suppl 1 S25-32.
Sun P, Sehouli J, Denkert C, Mustea A, Konsgen D, Koch I, Wei L and Lichtenegger W. Expression of estrogen receptor-related receptors, a subfamily of orphan nuclear receptors, as new tumor biomarkers in ovarian cancer cells. 2005; J Mol Med 83 (6) 457-467.
Suzuki T, Miki Y, Moriya T, Shimada N, Ishida T, Hirakawa H, Ohuchi N and Sasano H Estrogen-related receptor alpha in human breast carcinoma as a potent prognostic factor. 2004; Cancer Res 64 (13) 4670-4676.
Teyssier C G M, Rabier B, Monfoulet L, Dine J, Macari C, Espallergues J, Horard B, Giguere V, Cohen-Solal M, Chassande O, and Vanacker J M. Absence of ERRalpha in female mice confers resistance to bone loss induced by age or estrogen-deficiency. 2009; PLoS One 4 (11) 1-6.
Tremblay A M D C, Ghahremani M, Reudelhuber T L and Giguere V. Physiological genomics identifies estrogen-related receptor alpha as a regulator of renal sodium and potassium homeostasis and the renin-angiotensin pathway. 2010; Mol Endocrinol 24 (1) 22-32.
Vela I G L, Gardiner E M, Clements J A and Nicol D L. Bone and prostate cancer cell interactions in metastatic prostate cancer. 2007; BJU Int. 99 (4) 735-742.
Wang J, Fang F, Huang Z, Wang Y, Wong C. Kaempferol is an estrogen-related receptor alpha and gamma inverse agonist. FEBS Lett. 2009 Feb. 18; 583(4):643-7.
Wei W W X, Yang M, Smith L C, Dechow P C, Sonoda J, Evans R M and Wan Y. PGC1beta mediates PPARgamma activation of osteoclastogenesis and rosiglitazone-induced bone loss. 2010; Cell Metab 11 (6) 503-516.
Weilbaecher K N G T and McCauley L K. Cancer to bone: a fatal attraction. 2011; Nat Rev Cancer 11 (6) 411-425.

1. A method for predicting the occurrence of bone metastases in a prostate cancer patient comprising i) determining the level of expression of ERRalpha in a prostate tumor sample obtained from the patient, ii) comparing the level determined at step i) with a predetermined reference value and iii) concluding that there is a high risk that the patient develops bone metastases when the level determined at step i) is higher than the predetermined reference value or concluding that there is a low risk that the patient develops bone metastases when the level determined at step i) is lower than the predetermined reference value.

2. A method for the treatment of bone metastases in a prostate cancer patient in need thereof comprising administering to the patient an therapeutically effective amount of an inhibitor of ERRalpha activity or expression

3. The method according to claim 2 wherein the inhibitor of ERRalpha activity is an inverse agonist.

4. A method for screening a plurality of candidate compounds useful for treating or preventing bone metastases comprising the steps of (a) testing each of the candidate compounds for its ability to inhibit ERRalpha activity or expression and (b) positively selecting the candidate compounds capable of inhibiting said ERRalpha activity or expression.

5. The method of claim 3, wherein the inverse agonist is XCT-790.
